(19) United States
(12) Patent Application Publication
(10) Pub. No.: US 2025/0135001 A1
(43) Pub. Date: May 1, 2025

(54) COMPOSITIONS AND METHODS FOR CELLULAR IMMUNOLOGY

(71) Applicant: CARSGEN LIFE SCIENCES CO., LTD., Shanghai (CN)

(72) Inventors: Zonghai LI, Shanghai (CN); Zhaohui LIAO, Shanghai (CN); Qingzhou JI, Shanghai (CN); Shuang CHEN, Shanghai (CN)

(73) Assignee: CARSGEN LIFE SCIENCES CO., LTD., Shanghai (CN)

(21) Appl. No.: 18/837,379

(22) PCT Filed: Feb. 9, 2023
(86) PCT No.: PCT/CN2023/075205

§ 371 Date: Aug. 9, 2024

(30) Foreign Application Priority Data
Feb. 9, 2022 (CN) 202210122303.5
Feb. 11, 2022 (CN) 202210130437.1
Apr. 25, 2022 (CN) 202210443135.X
Apr. 27, 2022 (CN) 202210495909.3
Sep. 26, 2022 (CN) 202211178050.X

(51) Int. Cl. A61K 40/42; A61K 40/11; A61K 40/22; A61K 40/31; A61K 40/50; A61P 35/00; C07K 14/725; C07K 16/28; C12N 9/22; C12N 15/11; C12N 15/90

(52) U.S. Cl. CPC A61K 40/421; A61K 40/11; A61K 40/22; A61K 40/31; A61K 40/4215; A61K 40/50; A61P 35/00; C07K 14/7051; C07K 16/2803; C07K 16/2878; C12N 9/22; C12N 15/11; C12N 15/907; C12N 2310/20

(57) ABSTRACT

The present invention relates to a bispecific molecule targeting NK cells, and relates to a method for resisting transplant immune rejection caused by NK cells, and particularly relates to a method for providing antibodies targeting NK cells or for providing cells which secrete the antibodies targeting NK cells, so as to resist transplant immune rejection caused by NK cells of an individual receiving a transplant. The present invention also relates to a CRISPR/Cas-related methods, compositions, and components for editing a target nucleic acid sequence or modulating the expression of a target nucleic acid sequence.

### RELATED PATENT APPLICATIONS

This patent application claims the priority of the Chinese patent application with application number CN202210122303.5 filed on Feb. 9, 2022, the priority of the Chinese patent application with application number CN202210130437.1 filed on Feb. 11, 2022, the priority of the Chinese patent application with application number CN202210495909.3 filed on Apr. 27, 2022, the priority of the Chinese patent application with application number CN202211178050.X filed on Sep. 26, 2022, and the priority of the Chinese patent application with application No. 202210443135.X filed on Apr. 25, 2022.

### SEQUENCE LISTING SUBMITTED SIMULTANEOUSLY

The entire contents of the following XML file are incorporated herein by reference in their entirety: Sequence Listing in Computer Readable Format (CRF) (Name: FF00739PCT-sequence listing.xml, Date: 20230209, Size: 68.5 KB).

### TECHNICAL FIELD

This application belongs to the field of biotechnology. More particularly, the present application relates to a bispecific molecule targeting NK cells, and to a method for resisting transplant immune rejection caused by NK cells, and in particular to a method for resisting transplant immune rejection caused by NK cells of an individual receiving a transplant through administration of an antibody targeting NK cells or administration of a cell secreting an antibody targeting NK cells. The present application further relates to a CRISPR/CAS related method, composition and component for editing a target nucleic acid sequence or modulating the expression of a target nucleic acid sequence.

### BACKGROUND ART

Traditional immune cell therapy plays its role by activating, expanding or genetically modifying the autologous immune cells of a patent, and then infusing them into the patient. Due to the special technical characteristics, the autologous immune cell therapy are being troubled by some problems including high cost, non-availability, difficulty in scalability, and the inability to prepare the immune cells due to the deficiency of immune cells from the patient per se or the unsatisfactory treatment effects due to the poor quality of the prepared immune cells. Therefore, there is still a need to develop methods for preparing immune cells with stable quality that can be prepared on a large scale and be readily available.

The above problems are expected to be overcome by genetically editing healthy human T cells through gene editing technology to prepare allogeneic T cells. The first problem to be overcome in the preparation of allogeneic T cells is the attack of allogeneic T cells on host cells. Currently, there is a relatively mature method in which the TCR receptors of allogeneic T cells are knocked out to avoid graft-versus-host reaction (GvHD). In addition, the main factor affecting the durability and then the efficacy of allogeneic T cells in the host is the host rejection of allogeneic cells (HvDR). To address this problem, there are currently two types of strategies: the first strategy is to eliminate T cells in the host that may reject allogeneic cells. This strategy targets the host T cells, but the long-term absence of host T cells or activated T cells will seriously affect the host's own immune system. The second strategy is to eliminate the major histocompatibility antigen of allogeneic T cells, and the commonly used method is to knock out the B2M of allogeneic T cells. The knockout of B2M prevents the diverse enriched HLA-ABC proteins from being expressed on the cell membrane, thereby avoiding attacks from host T cells. However, the absence of HLA-I class molecules will lead to the elimination of HLA-I class molecule-deficient cells by host NK cells. Therefore, in order to confer allogeneic T cells an ability to survive longer in the body and thus better exert their anti-tumor effects, there is an urgent need to develop new strategies to resist the elimination of allogeneic T cells by the T cells or NK cells of a host.

The core problem for allogeneic CAR-T cells is how to avoid graft-versus-host (GVHD) reaction and host immune system rejection reaction (HVGR). GVHD may be avoided by knocking out TCR, immune rejection of allogeneic CD8 T cells may be avoided by knocking out HLA-I, and immune rejection of allogeneic CD4 T cells may be avoided by knocking out HLA-II. However, the absence of HLA-I significantly activates allogeneic NK cells, thereby enhancing NK cell immune rejection.

Direct delivery of the Cas9 ribonucleoprotein (RNP) complex allows for efficient gene editing, while minimizing off-target activity due to the rapid turnover of the Cas9 protein in cells. The efficiency of gene editing mediated by RNP delivery varies by locus, and depends on the length of gRNA selected, as well as the amount and ratio of Cas9 protein and gRNA delivered. However, low gene editing efficiency still exists in the gene editing process. Therefore, it is important for the knockout efficiency of a specific target gene to seek for a target sequence that can efficiently knock out the gene.

### SUMMARY

In a first aspect, the present application provides the technical solutions as described in items 1 to 23 below.
1. A bispecific molecule, wherein the molecule comprises: a first binding domain that binds to a NK cell receptor on the surface of a target cell; and a second binding domain that binds to a CD3 on the surface of a T cell.
2. The molecule according to item 1, wherein the NK cell receptor comprises a NK inhibitory receptor and/or a NK activating receptor.
3. The molecule according to item 1 or 2, wherein the NK cell receptor comprises NKG2A and/or NKP46.
4. The molecule according to any one of items 1-3, wherein the first binding domain binds to human or macaque NKG2A and/or NKP46; and/or the second binding domain binds to human CD3ε, Callithrix jacchus CD38, Saguinus oedipus CD3ε, or Saimiri sciureus CD3ε.
5. The molecule according to any one of items 1-4, wherein the molecule is any one selected from the group consisting of: scFv, (scFv)₂, scFv-single domain antibody, bifunctional antibody, and an oligomer thereof.
6. The molecule according to any one of items 1-5, wherein the first binding domain comprises a sequence represented by SEQ ID NO: 34 and SEQ ID NO: 35; the second binding domain comprises a sequence represented by SEQ ID NO: 44 and SEQ ID NO: 45.
7. The molecule according to any one of items 1-6, wherein the molecule comprises a nucleic acid sequence capable of expressing the amino acid sequence represented by SEQ ID NO: 59 and/or 63; or comprises the amino acid sequence represented by SEQ ID NO: 59 and/or 63.
8. A nucleic acid encoding a molecule according to any one of items 1-7.
9. A vector comprising the nucleic acid according to item 8.
10. An immune cell transformed or transfected with the nucleic acid according to item 8 or with the vector according to item 9.
11. The immune cell according to item 10, wherein the immune cell can secrete the molecule according to any one of items 1-7.
12. The immune cell according to item 10 or 11, wherein the immune cell further expresses a ligand or antibody fragment of a membrane-bound NK cell inhibitory receptor.
13. The immune cell according to any one of items 10-12, wherein the immune cell further expresses a membrane-bound NKG2A antibody or antibody fragment.
14. The immune cell according to any one of items 10-13, wherein the endogenous NKG2A gene of the immune cell is knocked out, preferably the endogenous NKG2A gene of the immune cell is knocked out by CRISPR/Cas9 technology.
15. The immune cell according to any one of items 10-14, wherein the cell further expresses a non-NKG2A-targeted chimeric antigen receptor, and the non-NKG2A-targeting chimeric antigen receptor recognizes a tumor antigen or a pathogen antigen; preferably, the tumor antigen comprises BCMA, CD19, GPC3, Claudin18.2, EGFR, EGFRVIII, or a combination thereof.
16. The immune cell according to any one of items 10 to 15, wherein the cell is derived from a natural T cell and/or a T cell induced by pluripotent stem cell;
preferably, the T cell is an autologous/allogeneic T cell;
preferably, the T cell a primary T cell; and
preferably, the T cell is derived from a human autologous T cell.
17. The immune cell according to any one of items 10-16, wherein the T cell comprises memory stem cell-like T cell (Tscm cell), central memory T cell (Tcm), effector T cell (Tef), regulatory T cell (Treg), effector memory T cell (Tem), γδT cell or a combination thereof.
18. The immune cell according to any one of items 10-17, wherein the endogenous MHC and endogenous TCR of the immune cell are knocked out, preferably by CRISPR/Cas9 technology.
19. A pharmaceutical composition, which comprises the molecule according to any one of items 1 to 7, the nucleic acid according to item 8, the vector according to item 9, or the immune cell according to any one of items 10 to 18; or further comprises T cells expressing a non-NKG2A-targeting chimeric antigen receptor,
preferably, the non-NKG2A-targeting chimeric antigen receptor targets a tumor or pathogen antigen,
more preferably, the non-NKG2A-targeting chimeric antigen receptor targets BCMA, CD19, GPC3, Claudin18.2, EGFR, EGFRVIII, or a combination thereof.
20. A method for producing the molecule according to any one of items 1-7, wherein the method comprises culturing the immune cell according to any one of items 10-18 under conditions allowing expression of the molecule according to any one of items 1-7, and recovering the produced molecule from the culture.
21. The molecule according to any one of items 1-7 or the molecule produced by the method according to item 20, for use to increase the durability and/or transplant survival rate of immune cells in the presence of host NK cells.
22. A method for increasing the durability and/or transplant survival rate of allogeneic immune cells in the presence of host NK cells, which comprises administering to a subject in need thereof the molecule according to any one of items 1-7, the molecule produced by the method according to item 19, the nucleic acid according to item 8, the vector according to item 9, and/or the immune cell according to any one of items 10-18.
23. A kit comprising: the molecule according to any one of items 1-7, the molecule produced by the method according to item 19, the immune cell according to any one of items 10-18, the nucleic acid according to item 8, and/or the vector according to item 9.

The second aspect of the present application provides the technical solutions described in the following items (1) to (20).
(1). A gRNA construct comprising a first gRNA targeting the CIITA gene, wherein the fragment comprises a nucleotide sequence represented by SEQ ID NO: 1, 2, 3, 4, 5, 6, 7, 8, 9, 10, 11, 12 or 13.
(2). The construct according to item (1), further comprising a second gRNA targeting the TRAC gene and/or a third gRNA targeting the B2M gene.
(3). The construct according to item (2), wherein the second gRNA comprises the nucleotide sequence represented by SEQ ID NOs: 24, 64 and/or 65; and/or the third gRNA comprises the nucleotide sequence represented by SEQ ID NOs: 25, 66 and/or 67.
(4). The construct according to item (3), wherein it comprises:
the first gRNA having a nucleotide sequence represented by SEQ ID NO: 4, the second gRNA having a nucleotide sequence represented by SEQ ID NO: 24, and the third gRNA having a nucleotide sequence represented by SEQ ID NO: 25;
the first gRNA having a nucleotide sequence represented by SEQ ID NO: 4, the second gRNA having a nucleotide sequence represented by SEQ ID NO: 64, and the third gRNA having a nucleotide sequence represented by SEQ ID NO: 25;
the first gRNA having a nucleotide sequence represented by SEQ ID NO: 4, the second gRNA having a nucleotide sequence represented by SEQ ID NO: 24, and the third gRNA having a nucleotide sequence represented by SEQ ID NO: 66;
the first gRNA having a nucleotide sequence represented by SEQ ID NO: 4, the second gRNA having a nucleotide sequence represented by SEQ ID NO: 65, and the third gRNA having a nucleotide sequence represented by SEQ ID NO: 66; and/or
the first gRNA having a nucleotide sequence represented by SEQ ID NO: 4, the second gRNA having a nucleotide sequence represented by SEQ ID NO: 24, and the third gRNA having a nucleotide sequence represented by SEQ ID NO: 67.
(5). The construct according to any one of items (1) to (4), comprising any first gRNA connected to crRNA/tracrRNA; further comprising any second gRNA connected to crRNA/tracrRNA, and/or further comprising any third gRNA fragment connected to crRNA/tracrRNA.
(6). The construct according to item (5), wherein the crRNA/tracrRNA comprises a nucleotide sequence represented by SEQ ID NO: 26.
(7). A method for gene editing of CIITA in a cell by the CRISPR/Cas system, wherein the gene editing on the cell is performed by using the construct according to any one of the above items (1)-(6).
(8). The method according to item (7), wherein the Cas enzyme is Cas9 enzyme.
(9). The method according to item (8), wherein the enzymatic activity of the Cas9 enzyme is 0.1-1 nmol, preferably 0.2-0.7 nmol, and more preferably 0.3-0.5 nmol.
(10). A method according to any one of items (7)-(9), wherein it comprises simultaneously introducing a complex of a nucleic acid and a protein comprising a Cas protein mixed with a gRNA according to any one of items (1)-(6) into the cell for gene editing.
(11). The method according to any one of items (7) to (10), wherein a molar ratio of the Cas9 enzyme to the total gRNA comprising the gRNA according to any one of items (1) to (6) is 1:1 to 1:10, preferably 1:3 to 1:5, and further preferably 1:4.
(12). The method according to any one of items (7) to (11), wherein the molar ratio of the first gRNA to the second gRNA is about 1:5 to 5:1, preferably 1:2 to 2:1; and more preferably about 1:1.
(13). The method according to any one of items (7) to (12), wherein the molar ratio of the first gRNA to the third gRNA is about 1:5 to 5:1, preferably 1:2 to 2:1; and more preferably about 1:1.
(14). The method according to any one of items (7) to (13), wherein the molar ratio of the Cas9 enzyme and the gRNA is 1:1 to 1:10, preferably 1:3 to 1:5, and more preferably 1:4, in the first complex formed by the Cas9 enzyme and the first gRNA, the second complex formed by the Cas9 enzyme and the second gRNA, and the third complex formed by the Cas9 enzyme and the third gRNA.
(15). The method according to item (14), wherein the concentration of the Cas9 enzyme in the first complex, or the second complex, or the third complex is about 0.1 μM-3 μM; preferably about 0.125 μM-3 μM; more preferably about 0.2 μM-3 μM; more preferably about 0.25 μM-3 μM; more preferably about 0.5 μM-3 μM; more preferably about 1 μM-3 μM.
(16). The method according to any one of items (7) to (15), wherein the cell is an eukaryotic cell.
(17). The method according to any one of items (7) to (16), wherein the cell is a T cell or pluripotent stem cell.
(18). A cell, which is constructed by the method according to any one of items (7) to (17).
(19). An allogeneic T cell, which is constructed by the method according to any one of items (7) to (18).
(20). The T cell according to item (19), wherein the T cell further expresses a chimeric antigen receptor, preferably the T cell further expresses a chimeric receptor recognizing a tumor antigen or a pathogen antigen; the chimeric receptor comprises an extracellular antigen binding domain, a transmembrane domain, and an intracellular domain, and wherein the extracellular antigen binding domain specifically recognizes a target antigen.

The third aspect of the present application provides the technical solutions as described in the following items (1) to (13).
(1). A gRNA construct comprising a gRNA, wherein the gRNA comprises a sequence represented by SEQ ID NO: 14 and/or SEQ ID NO: 15.
(2). The construct according to item (1), comprising the gRNA connected to crRNA/tracrRNA.
(3). The construct according to item (2), wherein the crRNA/tracrRNA comprises a nucleotide sequence represented by SEQ ID NO: 26.
(4). The construct according to any one of items (1) to (3), wherein it is used to knock out the NKG2A gene in the cell.
(5). A method for gene editing of NKG2A in a cell by the CRISPR/Cas system, which comprises performing gene editing on the cell by using the construct according to any one of items (1)-(4).
(6). The method according to item (5), wherein the Cas protein is selected from: Cas9 protein, Cas12a protein, Cas12b protein, Cas12c protein, Cas12d protein, Cas12e protein, Cas12f protein, Cas12g protein, Cas12h protein, Cas12i protein, Cas14 protein, Cas13a protein, Cas1 protein, Cas1B protein, Cas2 protein, Cas3 protein, Cas4 protein, Cas5 protein, Cas6 protein, Cas7 protein, Cas8 protein, Cas10 protein, Csy1 protein, Csy2 protein, Csy3 protein, Cse1 protein, Cse2 protein, Csc1 protein, Csc2 protein, Csa5 protein, Csn2 protein, Csm2 protein, Csm3 protein, Csm4 protein, Csm5 protein, Csm6 protein, Cmr1 protein, Cmr3 protein, Cmr4 protein, Cmr5 protein, Cmr6 protein, Csb1 protein, Csb2 protein, Csb3 protein, Csx17 protein, Csx14 protein, Csx10 protein, Csx16 protein, CsaX protein, Csx3 protein, Csx1 protein, Csx15 protein, Csfl protein, Csf2 protein, Csf3 protein, Csf4 protein, and a homologue or modified form thereof.
(7). The method according to item (6), wherein the enzymatic activity of the Cas9 enzyme is 0.1-1 nmol, preferably 0.2-0.7 nmol, and more preferably 0.3-0.5 nmol.
(8). A method according to any one of items (5)-(7), wherein it comprises simultaneously introducing a complex of a nucleic acid and a protein comprising a Cas enzyme mixed with a gRNA according to any one of items (1)-(4) into the cell for gene editing.
(9). The method according to any one of items (5) to (8), wherein the cell is selected from: T cell, NK cell, cytotoxic T cell, NKT cell, macrophage, CIK cell, stem cell and stem cell-derived immune cell, or a combination thereof.
(10). The method according to any one of items (5) to (9), wherein the cell is selected from: autologous or allogeneic T cell, stem cell-derived T cell, primary T cell or autologous T cell derived from human.
(11). A cell constructed by the method according to any one of items (5) to (10).
(12). The cell according to item (11), wherein the cell further expresses an exogenous receptor, and preferably the cell further expresses a chimeric receptor recognizing a tumor antigen and/or pathogen antigen.
(13). A kit comprising the construct according to any one of items (1) to (4).

The fourth aspect of the present application provides the technical solutions as described in items 1 to 32 below.
1. A gRNA construct comprising a first gRNA, wherein the first gRNA comprises a sequence represented by SEQ ID NO: 1, 2, 4, 7, 8, 9, 10, 12 or 13.
2. The construct according to item 1, the first gRNA comprises a sequence of continuous 16, 17, 18 or 19 nucleotides in the sequence represented by SEQ ID NO: 1, 2, 4, 7, 8, 9, 10, 12 or 13.
3. The construct according to item 1 or 2, wherein the first gRNA targets the CIITA gene.
4. The construct according to any one of items 1-3, further comprising a second gRNA targeting the TRAC gene, a third gRNA targeting the B2M gene, and/or a fourth gRNA targeting NKG2A.
5. The construct according to item 4, wherein the second gRNA comprises a sequence represented by SEQ ID NOs: 24, 64 and/or 65; and/or the third gRNA comprises a sequence represented by SEQ ID NOs: 25, 66 and/or 67; and/or the fourth gRNA comprises a sequence represented by SEQ ID NOs: 14, 15 and/or 23.
6. The construct according to item 5, wherein the first, second, third and fourth gRNAs comprise the sequences represented by SEQ ID NOs: 4, 24, 25 and 23, respectively; or the first, second, third and fourth gRNAs comprise the sequences represented by SEQ ID NOs: 12, 24, 25 and 23, respectively; or the first, second, third and fourth gRNAs comprise the sequences represented by SEQ ID NOs: 13, 24, 25 and 23, respectively.
7. The construct according to any one of items 1-6, which comprises the first gRNA, the second gRNA, the third gRNA, or the fourth gRNA connected to crRNA/tracrRNA, respectively.
8. The construct according to item 7, wherein the crRNA/tracrRNA comprises a sequence represented by SEQ ID NO: 26
9. A method for gene editing of CIITA in a cell by the CRISPR/Cas system, which comprises performing gene editing on the cell by using the construct according to any one of items 1-8.
10. The method according to item 9, wherein the Cas enzyme is Cas9 enzyme.
11. The method according to item 10, wherein the enzymatic activity of the Cas9 enzyme is 0.1-1 nmol, preferably 0.2-0.7 nmol, and further preferably 0.3-0.5 nmol.
12. A method according to any one of items 9-11, wherein it comprises simultaneously introducing a complex of a nucleic acid and a protein comprising a Cas enzyme mixed with the gRNA according to any one of items 1-8 into the cell for gene editing.
13. The method according to any one of items 9 to 12 wherein the molar ratio of the Cas9 enzyme and the gRNA according to any one of items 1 to 8 to the total gRNA is 1:1-1:10, preferably 1:3-1:5, and further preferably 1:4.
14. The method according to any one of items 9 to 13, wherein the cell is selected from: T cell, NK cells, cytotoxic T cell, NKT cells, macrophage, CIK cell, stem cell, and stem cell-derived immune cell, or a combination thereof.
15. The method according to any one of items 9 to 14, wherein the cell is selected from: autologous or allogeneic T cell, stem cell-derived T cell, primary T cell or autologous T cell derived from human.
16. A cell constructed by the method according to any one of items 9-15.
17. The cell according to item 16, wherein the cell further expresses an exogenous receptor, preferably the cell further expresses an exogenous receptor recognizing an NKG2A polypeptide, a tumor antigen, and/or a pathogen antigen.
18. A cell comprising a knockout of a gene encoding HLA-I/TCR/CIITA/NKG2A protein, and/or with low expression or no expression of endogenous HLA-I/TCR/HLA-II/NKG2A molecules.
19. The cell according to item 18, wherein the endogenous TCR/B2M/CIITA/NKG2A molecule is knocked out by the CRISPR/Cas9 technology.
20. The cell according to item 18, wherein the cell is genetically modified by the construct according to items 1-8 or the method according to items 9-15.
21. The cell according to item 19, wherein the gRNA used in the CRISPR/Cas9 technology comprises a sequence represented by SEQ ID NOs: 4, 24, 25 and 23; or comprises a sequence represented by SEQ ID NOs: 12, 24, 25 and 23; or comprises a sequence represented by SEQ ID NOs: 13, 24, 25 and 23.
22. The cell according to any one of items 18-21, wherein the cell further expresses a exogenous receptor recognizing an NKG2A polypeptide, a tumor antigen and/or a pathogen antigen.
23. The cell according to item 22, wherein the exogenous receptor comprises a chimeric antigen receptor (CAR), a chimeric T cell receptor, a T cell antigen coupler (TAC), or a combination thereof.
24. The cell according to item 23, wherein the CAR comprises:
a) an antibody recognizing an NKG2A polypeptide, a tumor and/or a pathogen antigen, a transmembrane region of CD28 or CD8, a co-stimulatory signaling domain of CD28, and CD3δ; and/or
b) an antibody recognizing an NKG2A polypeptide, a tumor and/or a pathogen antigen, a transmembrane region of CD28 or CD8, a co-stimulatory signaling domain of CD137, and CD3δ; and/or
c) an antibody recognizing an NKG2A polypeptide, a tumor and/or a pathogen antigen, a transmembrane region of CD28 or CD8, a costimulatory signaling domain of CD28, a costimulatory signaling domain of CD137, and CD3δ; or
d) an antibody recognizing an NKG2A polypeptide, a tumor and/or a pathogen antigen, a transmembrane region of CD28 or CD8, and CD3δ.
25. The cell according to any one of items 18-24, wherein the cell is selected from: T cell, NK cell, cytotoxic T cell, NKT cell, macrophage, CIK cell, stem cell, and stem cell-derived immune cell, or a combination thereof.
26. The cell according to any one of items 18-25, wherein the cell is selected from: autologous or allogeneic T cell, stem cell-derived T cell, primary T cell, or autologous T cell derived from human.
27. The cell according to any one of items 22-26, wherein the tumor antigen is selected from: CD19, GPC3, Claudin18.2, WT1, HER2, EGFR, BCMA, or a combination thereof.
28. The cell according to any one of items 22-27, wherein the antibody recognizing the NKG2A polypeptide comprises: a heavy chain variable region represented by SEQ ID NO:34, and/or a light chain variable region represented by SEQ ID NO:35; or a tandem antibody sequence represented by SEQ ID NO:46, 47, 48, 49 or 50.
29. The cell according to any one of items 22-28, wherein the antibody recognizing the tumor antigen comprises: a heavy chain variable region represented by SEQ ID NO: 27, and/or a light chain variable region represented by SEQ ID NO:
28; or a scFv represented by SEQ ID NO: 29, 30, 31, 32 or 33; or a tandem antibody sequence represented by SEQ ID NO: 46, 47, 48, 49 or 50.
30. A pharmaceutical composition, which comprises an effective amount of: the construct according to any one of items 1 to 8, the cell according to any one of items 16 to 29, and a pharmaceutically acceptable excipient.
31. The pharmaceutical composition according to item 30, for use in the treatment or prevention of a tumor.
32. A kit comprising the construct according to any one of items 1-8, the cell according to any one of items 16-29, or the pharmaceutical composition according to item 30 or 31.

It should be understood that within the scope of the present application, the above-mentioned technical features of the present application and the technical features specifically described below (such as embodiments/examples) may be combined with each other to form new or preferred technical solutions. Due to space limitations, each of these features will not be detailed herein.

### BRIEF DESCRIPTION OF THE DRAWINGS

FIG. 1 shows the knockout efficiency of different CIITA-gRNAs;

FIG. 2 shows that the in vitro activation of allogeneic CD4+ T cells can be reduced by UCAR-T cells with endogenous TCR/B2M/CIITA/NKG2A knockout and recognition of BCMA tumor antigen;

FIG. 3 shows that in the presence of allogeneic immune cells, UCAR-T cells with endogenous TCR/B2M/CIITA/NKG2A knockout and recognition of BCMA tumor antigens exhibit better expansion and survival in vivo;

FIG. 4 shows that tumor cells can be killed in vitro by UCAR-T cells with endogenous TCR/B2M/CIITA/NKG2A knockout and recognition of BCMA tumor antigen;

FIG. 5 shows that tumor cells can be killed in vitro by tandem UCAR-T cells with endogenous TCR/B2M/CIITA/NKG2A knockout and recognition of NKG2A peptide and BCMA tumor antigen;

FIG. 6 shows that tandem UCAR-T cells with endogenous TCR/B2M/CIITA/NKG2A knockout and recognition of NKG2A peptide and BCMA tumor antigen can exert anti-tumor effects in vivo;

FIG. 7 shows that in the presence of NK cells, UCAR-T cells with endogenous TCR/B2M/CIITA/NKG2A knockout and recognition of NKG2A can both promote the in vitro survival and/or expansion of UCAR-T cells in the compositionand exert a synergistic anti-tumor effect;

FIG. 8A shows that in the presence of NK cells, the in vivo anti-tumor activity of UCAR-T cells can be promoted by UCAR-T cells with endogenous TCR/B2M/CIITA/NKG2A knockout and recognition of NKG2A; FIG. 8B shows that the in vivo expansion and survival of UCAR-T cells can be promoted by UCAR-T cells with endogenous TCR/B2M/CIITA/NKG2A knockout and recognition of NKG2A;

FIG. 9 shows that NK cells can be effectively lysed in vitro by T cells expressing an NKG2A-CD3 bifunctional antibody;

FIG. 10A shows that the proportion of NK cells in the co-culture system can be reduced by T cells expressing NKG2A-CD3/NKP46-CD3 bifunctional antibodies;

FIG. 10B shows that the proliferation of NK cells can be inhibited by the above T cells;

FIG. 11 shows that the proliferation of NK cells can be inhibited by T cells expressing different cloned forms of NKG2A-CD3 bifunctional antibodies;

FIG. 12 shows that the proliferation of NK cells can be inhibited by the culture supernatant containing the NKG2A-CD3/NKP46-CD3 bifunctional antibodies;

FIG. 13A shows that B2M knockout T cells expressing an NKG2A-CD3/NKP46-CD3 bifunctional antibody exhibit a higher survival when co-cultured with NK cells;

FIG. 13B shows that when co-cultured with NK cells, the above cells exhibit better survival and inhibit the proliferation of NK cells.

FIG. 14 shows that in the presence of both NK cells and tumor cells, the NK cell proliferation can be inhibited by T cells expressing NKG2A-CD3/NKP46-CD3 bifunctional antibodies;

FIG. 15 shows that in the presence of NK cells and tumor cells, the expansion and survival of UCAR-T cells can be promoted by T cells expressing NKG2A-CD3/NKP46-CD3 bifunctional antibodies;

FIG. 16 shows that in the presence of NK cells and tumor cells, the proliferation of NK cells can be inhibited by the culture supernatant containing the NKG2A-CD3/NKP46-CD3 bifunctional antibodies;

FIG. 17 shows that in the presence of NK cells and tumor cells, the expansion and survival of UCAR-T cells can be promoted by the culture medium supernatant containing an NKG2A-CD3/NKP46-CD3 bifunctional antibody.

### DETAILED DESCRIPTION

After extensive and in-depth research, the inventors unexpectedly found that NKG2A-CD3 bispecific molecules and/or NKP46-CD3 bispecific molecules can significantly enhance the killing of host NK cells and eliminate host NK cells, thereby increasing the durability and/or transplant survival rate of autologous or allogeneic T cells in the presence of host immune cells (such as NK cells).

Unless otherwise defined, all technical and scientific terms used herein have the same meanings as commonly understood by those skilled in the fields of gene therapy, biochemistry, genetics and molecular biology. All methods and materials similar or equivalent to those described herein can be used in the practice or testing of the present application, wherein appropriate methods and materials are described herein. All publications, patent applications, patents, and other references mentioned herein are incorporated by reference in their entirety. In case of conflict, the present specification, including definitions, will control. In addition, unless otherwise specified, the materials, methods, and examples are illustrative only and not intended to be limiting.

Unless otherwise indicated, the practice of the present application will employ conventional techniques of cell biology, cell culture, molecular biology, transgenic biology, microbiology, recombinant DNA, and immunology, which are within the scope of the skill of the art. Such techniques are explained fully in the literatures. See, e.g., Current Protocols in Molecular Biology (Frederick M. AUSUBEL, 2000, Wiley and son Inc, Library of Congress, USA); Molecular Cloning: A Laboratory Manual, Third Edition, (Sambrook et al, 2001, Cold Spring Harbor, New York: Cold Spring Harbor Laboratory Press); Oligonucleotide Synthesis (M. J. Gaited., 1984); Mullis et al. U.S. Pat. No. 4,683,195; Nucleic Acid Hybridization (B. D. Harries & S. J. Higginseds. 1984); Transcription And Translation (B. D. Hames & S. J. Higginseds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the series, Methods In ENZYMOLOGY (J. Abelson and M. Simon, eds.-in-chief, Academic Press, Inc., New York), especially Vols. 154 and 155 (Wu et al. eds.) and Vol. 185, “Gene Expression Technology” (D. Goeddel, ed.); Gene Transfer Vectors For Mammalian Cells (J. H. Miller and M. P. Calos eds., 1987, Cold Spring Harbor Laboratory); Immunochemical Methods In Cell And Molecular Biology (Mayer and Walker, eds., Academic Press, London, 1987); Hand book Of Experimental Immunology, Volumes I-IV (D. M. Weir and C. C. Blackwell, eds., 1986); and Manipulating the Mouse Embryo (Cold Spring Harbor Laboratory Press, Cold Spring Harbor, N.Y., 1986).

The subject matter claimed for protection in this application is presented in the form of a range. It should be understood that the description in the form of a range is only for convenience and brevity, and should not be interpreted as a rigid limitation on the scope of the subject matter claimed for protection. Accordingly, the description of a range should be considered to have specifically disclosed all the possible subranges, as well as individual numerical values within that range. For example, where a range is provided, it should be understood that the smaller ranges between the upper and lower limits of that range may independently comprise the upper and lower limits of those smaller ranges, and are also within the scope of the claimed subject matter, except where the upper and lower limits of the stated ranges are excluded expressly. When the stated range comprises one or both of the limits, the claimed subject matter also comprises ranges excluding one or both of those limits. This applies regardless of the width of the range.

The term “about” as used herein refers to the typical error range for each value that is readily known by one skilled in the art. Reference herein to “about” a value or parameter comprises embodiments referring to the value or parameter per se. For example, description of “about X” comprises description of “X”. Herein, “about” may be an acceptable error range in the technical field; for example, it may refer to a value or parameter within the range of ±10% of the “about” value or parameter, for example, about 5 uM may comprise any number between 4.5 uM and 5.5 uM.

Unless otherwise indicated, any concentration range, percentage range, ratio range or integer range described herein should be understood to comprise any integer within the range, and, where appropriate, fractional values thereof (e.g., tenths and hundredths of an integer).

### Terms

The term “NKG2A” (natural killer group 2A, also known as killer cell lectin like receptor C1) refers to an inhibitory receptor in the NKG2 lectin receptor family, which is mainly expressed on the surface of NK cells and some T cells (CD8+ T cells, Th2 cells, γδ T cells and NKT cells). The NCBI GenBank Gene ID of NKG2A is 3821, located at 12p13.2, with a start site of 10442264 (NC_000012.12) and a stop site of 10454685 (NC_000012.12). The NKG2A polypeptide comprises an amino acid sequence or a fragment thereof that is at least about 80%, at least about 85%, at least about 90%, at least about 95%, at least about 96%, at least about 97%, at least about 98%, at least about 99% or at least about 100% homologous or identical to the amino acid sequence encoded by the transcript expressed by the gene of NCBI GenBank Gene ID: 3821, and/or optionally may comprises up to one, or up to two, or up to three conservative amino acid substitutions.

The term “NKP46” refers to a natural cytotoxicity receptor (NCR) expressed only on the surface of NK cells, which is a unique marker of NK cells and usually plays its killing role when KIR/KLR loses the ability to recognize “itself”. The NCBI GenBank Gene ID of NKP46 is 9437, located at 19q13.42, with a start site of 54898198 (NC_000019.10) and a stop site of 54938208 (NC_000019.10).

The term “CIITA” (class II major histocompatibility complex transactivator), also known as type II transactivator, is a trans-acting factor that participates in initiating HLA-II class gene transcription by binding to specific transcription factors. The NCBI GenBank Gene ID of CIITA is 4261, located at 16p13.13, with a start site of 10866206 (NC_000016.10) and a stop site of 10943021 (NC_000016.10).

The term “BCMA antigen” or “BCMA” generally refers to B-cell maturation antigen, which belongs to the TNF receptor superfamily. BCMA can activate the proliferation and survival of B cells after the BCMA binding to its ligand. BCMA is specifically and highly expressed in plasma cells and multiple myeloma cells, but is not expressed in hematopoietic stem cells and other normal tissue cells. “BCMA” may be any variant, derivative or isoform of the BCMA gene or encoded protein. The NCBI GenBank Gene ID of BCMA is 608.

The term “activated immune cells” refers to a change in intracellular protein expression caused by signaling pathway, leading to the initiation of an immune response. For example, a signal transduction cascade is generated when CD3 molecules aggregate in response to the ligand binding and the immunoreceptor tyrosine-based activation motifs (ITAMs).

The term “nucleic acid” or “polynucleotide” refers to deoxyribonucleic acid (DNA) or ribonucleic acid (RNA) and a polymer thereof in single- or double-stranded form, and comprises any nucleic acid molecule encoding a polypeptide of interest or a fragment thereof. The nucleic acid molecule only needs to maintain substantial identity with the endogenous nucleic acid sequence, and does not need to be 100% homologous or identical to the endogenous nucleic acid sequence. A polynucleotide that is “substantial identity” with an endogenous sequence is typically capable of hybridizing to at least one strand of a double-stranded nucleic acid molecule. “Hybridization” refers to the pairing between complementary polynucleotide sequences or portions thereof to form double-stranded molecules under various stringent conditions. The term “homology” or “identity” refers to the subunit sequence identity between two polymer molecules, for example, between two nucleic acid molecules such as two DNA molecules or two RNA molecules, or between two polypeptide molecules. The term “substantial identity” or “substantial homology” refers to polypeptides or nucleic acid molecules that exhibit at least about 50% homology or identical to a reference amino acid sequence or nucleic acid sequence. In one example, such a sequence is at least about 60%, 65%, 70%, 75%, 80%, 85%, 90%, 95%, 99% or 100% homologous or identical to the amino acid or nucleic acid sequence used for comparison. Sequence identity can be measured by sequence analysis software (e.g., BLAST, BESTFIT, GAP or PILEUP/PRETTYBOX programs).

The term “disease” refers to any condition that damages or interferes with the normal function of cells, tissues or organs, such as a tumor (cancer) or infection by a pathogen. Refractory cancers include, but are not limited to, cancers that are insensitive to radiotherapy, relapse after radiotherapy, insensitive to chemotherapy, relapse after chemotherapy, and cancers that are insensitive to CAR-T therapy or relapse after treatment.

The terms “therapeutically effective amount”, “therapeutically effective”, “effective amount”, or “in an effective amount” are used interchangeably herein, and as described herein refer to an amount of a compound, formulation, substance or composition, pharmaceutical composition that is effective to achieve a particular biological result, for example, but not limited to, an amount or dose sufficient to promote a T cell response. An effective amount of immune cells refers to but is not limited to: the number of immune cells that can increase, enhance or prolong anti-tumor activity; the increase in the number of anti-tumor immune cells or the number of activated immune cells; the number of immune cells that promote IFN-γ secretion, tumor regression, tumor shrinkage, and tumor necrosis.

The term “endogenous” refers to a nucleic acid molecule or polypeptide and the like that comes from the organism itself.

The term “exogenous” refers to a nucleic acid molecule or polypeptide that is not endogenous in the cell, or is expressed at a level insufficient to achieve the function it has when overexpressed; it encompasses any recombinant nucleic acid molecule or polypeptide expressed in the cell, such as exogenous, heterologous, and overexpressed nucleic acid molecules and polypeptides.

The term “recognition” refers to selective binding to a target antigen. In the present application, the immune cells expressing the exogenous receptors can recognize cells expressing the antigens to which the exogenous receptors specifically bind.

The term “CAR” comprises an antigen binding domain, a transmembrane domain, and an intracellular signaling domain. The intracellular signaling domain comprises a primary signaling domain, and/or a co-stimulatory signaling domain. The antigen binding domain of the CAR may be derived from a murine, humanized monoclonal antibody, or fully human monoclonal antibody. The term CAR is not particularly limited to CAR molecules, but also comprises CAR variants. The CAR variants comprise split CARs, in which the antigen binding domain and intracellular signaling domain of CRA are present on two separate molecules.

The term “engineering” refers to changing the genetic substance within a cell or obtaining cell products at the cellular level or organelle level, through some engineering means by the application of the principles and methods of cell biology and molecular biology. An engineered cell may also refer to a cell comprising an added, deleted and/or altered gene.

The term “engineered cell” may refer to an engineered cell of human or non-human animal origin.

The term “specific binding” refers to an antibody or ligand that recognizes and binds to a binding partner (such as tumor antigen) protein present in a sample, but the antibody or ligand does not substantially recognize or bind to other molecules in the sample.

The term “tumor antigen” refers to an antigen that is newly emerged or is overexpressed during the development and progression of a hyperproliferative disease. For example, the hyperproliferative disease is a cancer/tumor. For example, the hyperproliferative disease may be a solid tumor antigen, for example, the hyperproliferative disease may also be hematological tumor antigen.

Any tumor antigen may be used in the tumor-related embodiments according to the application (“examples” and “embodiments” are used interchangeably herein). The antigen is expressed as a polypeptide or complete protein or a portion thereof. The tumor antigens of the present application include, but are not limited to: thyroid stimulating hormone receptor (TSHR); CD171; CS-1; C-type lectin-like molecule-1; ganglioside GD3; Tn antigen; CD19; CD20; CD22; CD30; CD70; CD123; CD138; CD33; CD44; CD44v7/8; CD3δ; CD44v6; B7H3 (CD276), B7H6; KIT (CD117); interleukin 13 receptor subunit alpha (IL-13Rα); interleukin 11 receptor alpha (IL-11Rα); prostate stem cell antigen (PSCA); prostate specific membrane antigen (PSMA); carcinoembryonic antigen (CEA); NY-ESO-1; HIV-1 Gag; MART-1; gp100; tyrosinase; mesothelin; EpCAM; proteinase serine 21 (PRSS21); vascular endothelial growth factor receptor, vascular endothelial growth factor receptor 2 (VEGFR2); Lewis (Y) antigen; CD24; platelet-derived growth factor receptor beta (PDGFR-β); stage-specific embryonic antigen-4 (SSEA-4); cell surface-associated mucin 1 (MUC1) and MUC6; epidermal growth factor receptor family and mutants thereof (EGFR, EGFR2, ERBB3, ERBB4, and EGFRvIII); neural cell adhesion molecule (NCAM); carbonic anhydrase IX (CAIX); LMP2; ephrin type A receptor 2 (EphA2); fucosyl GM1; sialyl Lewis adhesion molecule (sLe); ganglioside GM3 (aNeu5Ac (2-3) bDGalp (1-4) bDGlcp (1-1) Cer; TGS5; high molecular weight melanoma-associated antigen (HMWMAA); o-acetyl GD2 ganglioside (OAcGD2); folate receptor; tumor vascular endothelial marker 1 (TEMI/CD248); tumor endothelial marker 7-related (TEM7R); Claudin 6, Claudin18.2, Claudin18.1; ASGPR1; CDH16; 5T4; 8H9; αvβ6 integrin; B cell maturation antigen (BCMA); CA9; K light chain (kappa light chain); CSPG4; EGP2, EGP40; FAP; FAR; FBP; embryonic AchR; HLA-A1, HLA-A2; MAGEA1, MAGE3; KDR; MCSP; NKG2D ligand; PSC1; ROR1; Sp17; SURVIVIN; TAG72; TEM1; fibronectin; tenascin; carcinoembryonic variant of tumor necrosis zone; G protein-coupled receptor class C group 5 member D (GPRC5D); X chromosome open reading frame 61 (CXORF61); CD97; CD179a; anaplastic lymphoma kinase (ALK); polysialic acid; placenta-specificity 1 (PLAC1); the hexose portion of GloboH glycoceramide (GloboH); mammary differentiation antigen (NY-BR-1); uroplakin 2 (UPK2); hepatitis A virus cellular receptor 1 (HAVCRI); adrenergic receptor beta 3 (ADRB3); pannexin 3 (PANX3); G protein-coupled receptor 20 (GPR20); lymphocyte antigen 6 complex locus K9 (LY6K); olfactory receptor 51E2 (OR51E2); TCR gamma alternate reading frame protein (TARP); Wilms tumor protein (WT1); ETS translocation variant gene 6 (ETV6-AML); sperm protein 17 (SPA17); X antigen family member 1A (XAGE1); angiopoietin binding cell surface receptor 2 (Tie2); melanoma cancer testis antigen-1 (MAD-CT-1); melanoma cancer testis antigen-2 (MAD-CT-2) T-2); Fos-related antigen 1; p53 mutant; human telomerase reverse transcriptase (hTERT); sarcoma translocation breakpoints; melanoma inhibitor of apoptosis (ML-IAP); ERG (transmembrane protease serine 2 (TMPRSS2) ETS fusion gene); N-acetylglucosaminyltransferase V (NA17); paired box protein Pax-3 (PAX3); androgen receptor; cyclin B1; V-myc avian myelocytic virus oncogene neuroblastoma-derived homolog (MYCN); Ras homolog family member C (RhoC); cytochrome P450 1B1 (CYP1B1); CCCTC-binding factor (zinc finger protein)-like (BORIS); squamous cell carcinoma antigen recognized by T cells 3 (SART3); paired box protein Pax-5 (PAX5); proacrosin binding protein sp32 (OYTES1); lymphocyte-specific protein tyrosine kinase (LCK); A kinase anchoring protein 4 (AKAP-4); synovial sarcoma X breakpoint 2 (SSX2); CD79a; CD79b; CD72; leukocyte-associated immunoglobulin-like receptor 1 (LAIR1); Fc fragment of the IgA receptor (FCAR); leukocyte immunoglobulin-like receptor subfamily member 2 (LILRA2); CD300 molecule-like family member f (CD300LF); C-type lectin domain family 12 member A (CLEC12A); bone marrow stromal cell antigen 2 (BST2); mucin-like hormone receptor-like 2 comprising an EGF-like module (EMR2); lymphocyte antigen 75 (LY75); glypican-3 (GPC3); Fc receptor-like 5 (FCRL5); and immunoglobulin lambda-like polypeptide 1 (IGLL1).

The terms “individual” and “subject” are interchangeable, and comprise human and animals from other species, including but not limited to: human, mice, rats, hamsters and guinea pigs, rabbits, dogs, cats, sheep, pigs, goats, cows, horses, apes, and monkeys.

The term “isolated” means a state that is altered or removed from its natural state. For example, a nucleic acid or peptide naturally present in a living animal is not “isolated”, but the same nucleic acid or peptide partially or completely separated from coexisting substances in its natural state is “isolated”. An isolated nucleic acid or protein may exist in a substantially purified form, or may exist in a non-native environment, such as a host cell.

The terms “peptide”, “polypeptide”, and “protein” are used interchangeably, and refer to a compound composed of amino acid residues covalently linked by peptide bonds.

The term “transplant immune rejection” refers to an immunological response that after a host receives a transplant such as an allogeneic tissue, organ, or cell and the like, the foreign transplant as a “foreign component” is firstly recognized, and then attacked, destroyed and eliminated by the host immune system.

The term “transplant” refers to a biological material or preparation for implantation into a host, which is derived from an individual other than the host. The transplant may be derived from any animal source, such as mammalian origin, preferably human.

The term “host-versus-graft reaction (HVGR)” generally refers to: when performing transplantation with an exogenous transplant from a donor, the exogenous transplant from a donor will be recognized and attacked by the host immune cells (such as NK cells), thereby inhibiting or eliminating the transplant from a donor, due to the immunogenetic differences between the donor and the recipient (or host).

The term “graft-versus-host disease (GVHD)” generally refers to: after the donor T lymphocytes recognize antigens on the host normal tissues, they will proliferate and release a series of cytokines to attack the host cells, due to the diversity of TCRs of exogenous transplanted donor T lymphocytes and incompatibility with the host HLA molecules.

The term “MHC” refers to a major histocompatibility complex, which is a general term for a population of genes encoding biocompatibility complex antigens. In human cells, MHC is called HLA antigen, and plays an important role in transplantation response, with rejection mediated by T cells that respond to histocompatibility antigens on the surface of the implanted tissue. HLA-I is composed of a heavy chain (a chain) and a light chain β2-microglobulin (B2M).

The term “increase of durability and/or transplant survival rate” means that during the course of treatment, the engineered cells administered to a subject remain in the subject for a longer time, and/or maintain a higher number in the subject as compared to a subject administered with non-engineered cells.

The term “allogeneic cells” refers to cells or cell populations used to treat a subject, which originate from a different individual of the same species.

The term “antibody” is generally refers to a immunoglobulin molecule or the immunologically active portion of an immune molecule, i.e., a molecule that comprises an antigen binding site specifically binding (“immunoreact”) to an antigen, which may comprise an intact antibody molecule (also known as immunoglobulin) or a fragment thereof retaining the antigen-binding ability. Examples of antibody fragments, antibody variants or binding domains comprise: (1) a Fab fragment, which is a monovalent fragment with VL, VH, CL and CHI domains; (2) a F(ab′)₂ fragment, which is a bivalent fragment with two Fab fragments connected by a disulfide bridge at the hinge region; (3) a Fd fragment with two VH and CHI domains; (4) a Fv fragment with the VL and VH domains of a single arm of an antibody; (5) a dAb fragment (Ward et al. (1989) Nature 341:544-546) with a VH domain; (6) isolated complementarity determining regions (CDRs); and (7) single-chain Fv (scFv), the latter is preferred (e.g., derived from a scFV library).

Exemplarily, the present application provides: an anti-BCMA antibody comprising a VH represented by SEQ ID NO: 27 and a VL represented by SEQ ID NO: 28; an anti-BCMA antibody comprising a scFv sequence represented by SEQ ID NO: 29, 30, 31, 32 or 33; an anti-NKG2A antibody comprising a VH represented by SEQ ID NO: 34 and a VL represented by SEQ ID NO: 35; an anti-NKG2A antibody comprising a VH represented by SEQ ID NO: 36 and a VL represented by SEQ ID NO: 37; an anti-NKG2A antibody comprising a VH represented by SEQ ID NO: 38 and a VL represented by SEQ ID NO: 39; an anti-NKG2A antibody comprising a VH represented by SEQ ID NO: 40 and a VL represented by SEQ ID NO: 41; an anti-NKP46 antibody comprising a VH represented by SEQ ID NO: 42 and a VL represented by SEQ ID NO: 43; an anti-CD3 antibody comprising a VH represented by SEQ ID NO: 44 and a VL represented by SEQ ID NO: 45; and a BCMA-NKG2A tandem antibody comprising a sequence represented by NO: 46, 47, 48, 49 or 50.

The term “bispecific molecule” refers to a molecule consisting of only one polypeptide chain as well as a molecule consisting of more than one polypeptide chain, wherein the chains may be identical (a homodimer, homotrimer, or homooligomer) or different (a heterodimer, heterotrimer or heterooligomer). The bispecific molecules of the present application may be composed of polypeptides, antibodies, antibody fragments such as scFv, Fab, and nanobodies.

The term “bispecific T cell engage antibody (BiTE)” refers to an antibody exhibiting dual binding specificity to two different antigens or two different epitopes, which comprises a bispecific antibody specifically binding to different epitopes of an antigen, and a bispecific and multispecific antibody binding to more than one antigen structure (e.g., two or three). The bispecific T cell engage antibody comprises: full-length monoclonal antibody, recombinant antibody, chimeric antibody, deimmunized antibody, humanized antibody and human antibody; and comprises a fragment of an antibody (such as VH, VHH, VL, (s) dAb, Fv, Fd, Fab, Fab′, F(ab′) 2 or “rIgG” (“half antibody”)); it also comprises a modified fragment of an antibody, also called an antibody variant, such as scFv; di-scFv or bi(s)-scFv; scFv-Fc; scFv-zipper; scFab; Fab2; Fab3; diabody; single-chain diabody; tandem diabody (Tandab); tandem di-scFv; tandem tri-scFv; and “minibody”. It is exemplified by the following structures: (VH-VL-CH3)₂, (scFv-CH3)₂, ((scFv)₂-CH3+CH3), ((scFv)₂-CH3), or (scFv-CH3-scFv)₂; multifunctional antibody, such as trifunctional antibody or tetrafunctional antibody; and single domain antibody, such as nanobody, or single variable domain antibody which comprises only one variable domain (which may be VHH, VH or VL) specifically binding to an antigen or epitope and being independent of other V regions or domains.

### BiTE Targeting NK Cells

The present application provides a bispecific antibody BiTE that targets both NK cells and T cells. The BiTE comprises a first binding domain that targets NK cells and a second binding domain that targets T cells.

In one example, the BiTE targets NKG2A. In one example, the BiTE targets NKP46. In one example, the BiTE targets both NKG2A and CD3. In one example, the BiTE targets both NKP46 and CD3. A T cell expressing BiTE is also known as T-BiTE cell. In one example, NKG2A-BiTE is tandemly composed of a single-chain antibody (scFv) targeting NKG2A and a single-chain antibody (scFv) targeting CD3. In one example, NKP46-BiTE is tandemly composed of a single-chain antibody (scFv) targeting NKP46 and a single-chain antibody (scFv) targeting CD3. In one example, a single chain antibody (scFv) targeting NKG2A or NKP46 and a single chain antibody (scFv) targeting CD3 are linked by a hinge. In one example, the hinge comprises GGGGS. In one example, the BiTE gene is constructed into the viral packaging plasmid pWPT, PRRLsin or an eukaryotic expression plasmid.

In one example, the first binding domain of NKG2A-BiTE comprises: a sequence represented by SEQ ID NO: 34 and/or SEQ ID NO: 35, or a sequence represented by SEQ ID NO: 36 and/or SEQ ID NO: 37, or a sequence represented by SEQ ID NO: 38 and/or SEQ ID NO: 39, or a sequence represented by SEQ ID NO: 40 and/or SEQ ID NO: 41; and/or the second binding domain of NKG2A-BiTE comprises: a sequence represented by SEQ ID NO: 44 and SEQ ID NO: 45.

In one example, the first binding domain of NKP46-BiTE comprises: the sequence represented by SEQ ID NO: 42 and/or SEQ ID NO: 43; and/or the second binding domain of NKP46-BiTE comprises the sequence represented by SEQ ID NO: 44 and SEQ ID NO: 45.

In one example, the BiTE comprises the sequences represented by SEQ ID NOs: 59, 60, 61, 62 and/or 63.

The sequences provided in the present application are not limited to the BiTEs comprising specific amino acid sequences represented by SEQ ID NOs: 59, 60, 61, 62 and/or 63. BiTEs comprising the amino acid sequences that are modified, and/or changed by one or more amino acids of substitution, and/or deletion, and/or addition based on the amino acid sequences represented by SEQ ID NOs: 59, 60, 61, 62 and/or 63, and have 60%, 65%, 70%, 75%, 80%, 85%, 90%, 95% or more identity with and have the same function as the amino acid sequences represented by SEQ ID NOs: 59, 60, 61, 62 and/or 63 are also within the scope of protection of the present application.

The BiTE provided in this application may be used to kill NK cells. BiTEs targeting NK cells may enhance the survival and proliferation of T cells and/or CAR-T cells that are introduced into a subject previously, simultaneously or subsequently, and may also enhance the killing of tumors and/or pathogens by T cells and/or CAR-T cells that are introduced into a subject previously, simultaneously or subsequently.

The present application provides a method for increasing the durability and/or transplant survival rate of engineered cells in the presence of host immune cells (such as NK cells) by using BiTEs targeting NK cells. In one example, TCR/B2M, TCR/B2M/HLA-II, TCR/B2M/NKG2A, and TCR/B2M/HLA-II/NKG2A in the engineered cells are lowly expressed or not expressed.

The present application provides a composition comprising BiTEs targeting NK cells and engineered cells. In one example, the endogenous HLA-II, TCR, HLA-I or NKG2A of the engineered cells in the composition are lowly expressed or not expressed. In one example, the endogenous B2M, CIITA, TCR, or NKG2A of the engineered cells in the composition are lowly expressed or not expressed. In one example, the composition comprises engineered cells with low expression or no expression of TCR/B2M, TCR/B2M/HLA-II, TCR/B2M/NKG2A, or TCR/B2M/HLA-II/NKG2A.

The present application provides an engineered cell expressing NKG2A-CD3 and/or expressing NKP46-CD3, and also provides a method for preparing the engineered cell and use of the engineered cell in killing NK cell. The present application provides a method for increasing the durability and/or transplant survival rate of engineered cells in the presence of host immune cells (e.g., NK cells). In one example, cells are engineered to express NKG2A-CD3 and/or NKP46-CD3. In one example, the cells are engineered to express NKG2A-CD3 and/or NKP46-CD3, thus reducing or eliminating the expression or activity of endogenous NKG2A. Optionally, the cells are also engineered to express an NKG2A binding protein, preferably an NKG2A membrane-bound antibody. In one example, the cells are engineered to express an NKG2A-CD3 and/or NKP46-CD3, thus reducing or eliminating the expression or activity of endogenous NKG2A; the cells are also engineered to express exogenous receptors (CAR, recombinant TCR receptors) targeting tumors and/or pathogens. In one example, the cells are engineered to express an NKG2A-CD3 and/or NKP46-CD3, thereby reducing or eliminating the expression or activity of B2M, CIITA and TCR; the cells are also engineered to express exogenous receptors (CAR, recombinant TCR receptors) targeting tumors and/or pathogens. In one example, the cells are engineered to express an NKG2A-CD3 and/or NKP46-CD3, thereby reducing or eliminating the expression or activity of B2M, NKG2A and TCR; the cells are also engineered to express exogenous receptors (CAR, recombinant TCR receptors) targeting tumors and/or pathogens. In some embodiments, the cells are engineered to express an NKG2A-CD3 and/or NKP46-CD3, thereby reducing or eliminating the expression or activity of B2M and TCR; the cells are also engineered to express chimeric receptors (CAR, recombinant TCR receptors) targeting tumors and/or pathogens. In one example, the cells are engineered to express an NKG2A-CD3 and/or NKP46-CD3, thereby reducing or eliminating the expression or activity of B2M, CIITA, TCR and NKG2A; the cells are also genetically engineered to express exogenous receptors (CAR, recombinant TCR receptors) targeting tumors and/or pathogens, and are also genetically engineered to express NKG2A binding proteins, preferably NKG2A membrane-bound antibodies.

The above-mentioned engineered cells may be used to kill NK cells. The engineered cells may enhance the survival and proliferation of T cells and/or CAR-T cells that are introduced into a subject previously, simultaneously or subsequently; and may also enhance the killing of tumors and/or pathogens by T cells and/or CAR-T cells that are introduced into a subject previously, simultaneously or subsequently.

The supernatant of the culture medium of the above-mentioned engineering cells may be used to kill NK cells. The supernatant of the culture medium of the engineered cell may enhance the survival and proliferation of T cells and/or CAR-T cells that are introduced into a subject previously, simultaneously or subsequently, and may also enhance the killing of tumors and/or pathogens by T cells and/or CAR-T cells that are introduced into a subject previously, simultaneously or subsequently.

Immune cells with low or no expression of endogenous CIITA, NKG2A, TCR/B2M/CIITA, TCR/B2M/NKG2A or TCR/B2M/CIITA/NKG2A are prepared by the present application by gene knockout technology and/or gene silencing technology.

Gene knockout technologies comprise: Argonaute, CRISPR/Cas technology, ZFN technology, TALE technology, TALE-CRISPR/Cas technology, Base Editor technology, Prime editing (PE) technology and/or homing endonuclease technology. Gene silencing technologies include, but are not limited to: antisense RNA, RNA interference, microRNA-mediated translation inhibition, etc.

### Clustered Regularly Interspaced Short Palindromic Repeats (CRISPR) System

The system comprises a Cas (a protein capable of modifying DNA by using crRNA as its guide), a CRISPR RNA (a crRNA, which comprises a RNA that guide the Cas into the correct fragment of host DNA, and a region (usually in the form of a hairpin loop) that binds to tracrRNA, wherein the crRNA forms an active complex with Cas), a trans-activating crRNA (tracrRNA, which binds to crRNA and forms an active complex with the Cas), and an optional fragment of a DNA repair template (a DNA that directs the cellular repair process and allow insertion of a specific DNA sequence). In one example, the Cas molecule is selected from but not limited to: Cas9, Cas12a, cas12b, cas12c, cas12d, cas12e, cas12f, cas12g, cas12h, cas12i, cas14, Cas13a, Cas13b, Cas13c, Cas13d, Cas13e, Cas13f, Cas1, Cas1B, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, and homologs or modified forms thereof.

The terms Cas enzyme, CRISPR enzyme, CRISPR protein, Cas protein, and CRISPR Cas may be used interchangeably.

In one example, Cas is Cas9. The a Cas9 molecule and/or Cas9 polypeptide comprises naturally an occurring Cas9 molecule and Cas9 polypeptide, as well as an engineered, altered or modified Cas9 molecule or Cas9 polypeptide, which differs from a reference sequence (e.g., the most similar naturally occurring Cas9 molecule), for example, by at least one amino acid residue. Herein, it should be understood by those skilled in the art that, the molar ratio of the Cas9 enzyme and the gRNA to be introduced is calculated based on the above-mentioned Cas9 enzyme activity, while confirming the concentration of the Cas9 enzyme in the introduced complex. When the activity of the Cas9 enzyme changes, those skilled in the art may make a calculation based on the ratio determined herein according to the description of the activity in the instructions of different enzymes, so as to select the concentration of the Cas9 enzyme to be used, and its molar ratio to the gRNA. In one example, the final concentration of the Cas9 enzyme in the RNP is about 0.5 μM, 1 μM, 1.5 μM, 2 μM, 2.5 μM, 3 μM, 3.5 μM, 4 μM, 4.5 μM, 5 μM, 5.5 M, 6 μM, 6.5 μM, 7 μM, 7.5 μM, 8 μM, 8.5 μM, 9 μM, 9.5 M, or 10 μM.

It should be understood by those skilled in the art that in the present application, the molar ratio of the Cas9 enzyme and the gRNA to be introduced is calculated based on the above-mentioned Cas9 enzyme activity, while confirming the concentration of the Cas9 enzyme in the introduced complex. When the activity of the Cas9 enzyme changes, those skilled in the art may make a calculation based on the ratio determined herein according to the description of the activity in the instructions of different enzymes, so as to select the concentration of the Cas9 enzyme to be used, and its molar ratio to the gRNA.

In one example, the Cas enzyme is a nickase. In a preferred embodiment, the Cas9 is delivered to the cell in the form of mRNA, which allows transient expression of the enzyme, thereby reducing toxicity. Cas9 may also be delivered to cells in a nucleotide construct that encodes and expresses the Cas9 enzyme. Alternatively, Cas9 may be expressed under the control of an inducible promoter.

In one example, nucleic acid fragments may be delivered to target cells by CRISPR/Cas9 typically using plasmids or electroporation. In one example, a complex comprising a nucleic acid fragment and a recombinant protein such as a ribonucleoprotein complex (RNP) of gRNA and Cas9 may be delivered to target cells by CRISPR/Cas9 typically using plasmids or electroporation. The crRNA needs to be designed for each application, since it is the sequence that is recognized by the Cas9 and directly binds to target DNA in the cells. The crRNA and tracrRNA may be combined together to form a guide RNA (gRNA).

The gRNA construct refers to a molecule in which the structure and/or function is based on the structure and/or function of a gRNA. The gRNA sequence of the present application may be represented by the gRNA targeting domain sequence. In one example, the gRNA sequence is a sequence targeting DNA. In one example, the gRNA sequence is a nucleic acid sequence that is completely or partially complementary to the DNA sequence targeted by the gRNA. Complete complementarity is not required, provided that sufficient complementarity is existed to cause hybridization and promote formation of a CRISPR complex. In one example, the degree of complementarity between the gRNA and its corresponding target sequence is about or more than about 50%, 60%, 75%, 80%, 85%, 90%, 95%, 97.5%, 99% or more, when optimal alignments is performed by using a suitable alignment algorithm. In one example, the gRNA construct comprises a molecule of a complete Cas9 guide sequence formed by a gRNA sequence and crRNA/TracrRNA. In one example, the crRNA/TracrRNA sequence is represented by SEQ ID NO: 26. In one example, the gRNA construct comprises a gRNA-targeting domain comprising a nucleic acid sequence that is fully or partially complementary to a targeted DNA. In one example, the gRNA construct comprises a targeting domain that is fully or partially complementary to a target domain in or near the target location. In one example, the targeting domain comprises a nucleotide sequence represented by any one of SEQ ID NOs: 1, 2, 3, 4, 5, 6, 7, 8, 9, 10, 11, 12, 13, 14, 15, 16, 17, 18, 19, 20, 21, 22, 23, 24, 25, 64, 65, 66, and 67, or a combination thereof. In one example, the gRNA construct is a single molecule or a chimeric gRNA molecule. In one example, the targeting domain comprises a sequence of 16, 17, 18 or 19 consecutive nucleotides in the sequence represented by SEQ ID NO: 1, 2, 3, 4, 5, 6, 7, 8, 9, 10, 11, 12, 13, 14, 15, 16, 17, 18, 19, 20, 21, 22, 23, 24, 25, 64, 65, 66, or 67, respectively. In one example, the targeting domain comprises a sequence of 20, 21, 22, 23, 24, 25 or 26 consecutive nucleotides in SEQ ID NO: 1, 2, 3, 4, 5, 6, 7, 8, 9, 10, 11, 12, 13, 14, 15, 16, 17, 18, 19, 20, 21, 22, 23, 24, 25, 64, 65, 66, or 67, respectively. The gRNA sequence provided in the present application is not limited to the above-mentioned gRNA constructs comprising any one of the nucleotide sequences represented by SEQ ID NOs: 1, 2, 3, 4, 5, 6, 7, 8, 9, 10, 11, 12, 13, 14, 15, 16, 17, 18, 19, 20, 21, 22, 23, 24, 25, 64, 65, 66 and 67 respectively, and the gRNA constructs comprising the nucleotide sequences that are modified and/or changed by one or more nucleotides of substitution, and/or deletion, and/or addition on the basis of the nucleotide sequences represented by SEQ ID NOs: 1, 2, 3, 4, 5, 6, 7, 8, 9, 10, 11, 12, 13, 14, 15, 16, 17, 18, 19, 20, 21, 22, 23, 24, 25, 64, 65, 66, and 67, and have 90% or more identity with and have the same function as the nucleotide sequences represented by SEQ ID NOs: 1, 2, 3, 4, 5, 6, 7, 8, 9, 10, 11, 12, 13, 14, 15, 16, 17, 18, 19, 20, 21, 22, 23, 24, 25, 64, 65, 66, and 67 respectively are also within the scope of protection of the present application.

In one example, when a single endogenous gene is knocked out, the molar ratio of Cas9 enzyme to gRNA is 1:1-1:10, preferably 1:3-1:5; more preferably 1:4. In one example, when two or more endogenous genes are knocked out, the molar ratio of total Cas9 enzyme to total gRNAs (i.e., the sum of the amount of substance of two or more gRNAs) is 1:1-1:10, preferably 1:3-1:5; more preferably 1:4.

In the present application, after knocking out the TCR, B2M, NKG2A and/or CIITA genes of the cells by CRISPR/Cas9 technology, the cells with low or no expression of TCR, B2M, NKG2A and/or HLA-II are obtained by sorting.

In one example, the present application comprises a plasmid composed of a gRNA construct and a Cas9 gene. In one example, the methods provided herein comprise delivering one or more gRNA constructs and one or more Cas9 polypeptides or nucleic acid sequences encoding Cas9 polypeptides to a cell. In one example, one or more gRNA constructs, one or more Cas9 polypeptides, or nucleic acid sequences encoding Cas9 polypeptides are delivered by vectors (e.g., AAV, adenovirus, lentivirus), and/or particles and/or nanoparticles, and/or electroporation. In one example, crRNA and tracrRNA comprising the gRNA targeting domain are administered alone, or a complete RNA may be administered. The CRISPR/Cas9 transgene is delivered by vectors (e.g., AAV, adenovirus, lentivirus), and/or particles and/or nanoparticles, and/or electroporation.

Low expression or no expression of HLA-II, TCR, B2M or NKG2A refers to a decrease of at least 1%, at least 5%, at least 10%, at least 20%, at least 30%, at least 40%, at least 50%, at least 60%, at least 70%, at least 80%, at least 90%, at least 95%, at least 99% or 100% in the expression of HLA-II, TCR, B2M or NKG2A in the cells, respectively. More particularly, low expression or non-expression of HLA-II, TCR, B2M or NKG2A means that the content of HLA-II, TCR, B2M or NKG2A in the cells is reduced by at least 1%, at least 5%, at least 10%, at least 20%, at least 30%, at least 40%, at least 50%, at least 60%, at least 70%, at least 80%, at least 90%, at least 95%, at least 99% or 100%, respectively. The expression or content of the protein in the cell may be determined by any suitable method known in the art, such as ELISA, immunohistochemistry, Western Blotting or flow cytometry, by using specific antibodies for HLA-II, TCR, B2M or NKG2A.

The present application provides a nucleic acid molecule encoding a gRNA targeting endogenous CIITA. Exemplarily, the gRNA targeting CIITA comprises SEQ ID NO: 1, 2, 3, 4, 5, 6, 7, 8, 9, 10, 11, 12, or 13, or a combination thereof. Exemplarily, the gRNA targeting NKG2A comprises SEQ ID NO: 14, 15, 16, 17, 18, 19, 20, 21, 22, or 23, or a combination thereof. Exemplarily, the gRNA targeting TRAC comprises SEQ ID NO: 24, 64, or 65, or a combination thereof. Exemplarily, the gRNA targeting B2M comprises SEQ ID NO: 25, 66, or 67, or a combination thereof. In one example, immune cells with low or no endogenous TCR/B2M/HLA-II expression are prepared by gene knockout technology and/or gene silencing technology. In one example, immune cells with low or no endogenous TCR/B2M/NKG2A expression are prepared by gene knockout technology and/or gene silencing technology. In one example, immune cells with low or no endogenous B2M/HLA-II expression are prepared by gene knockout technology and/or gene silencing technology. In one example, immune cells with low or no endogenous TCR/HLA-II expression are prepared by gene knockout technology and/or gene silencing technology. In one example, immune cells with low or no endogenous TCR/B2M/HLA-II/NKG2A expression are prepared by gene knockout technology and/or gene silencing technology. In one example, the present application provides a nucleic acid molecule encoding a gRNA targeting the gene TRAC of a chain of the endogenous TCR. In one example, the gRNA construct of the present application comprises gRNAs targeting CIITA, NKG2A, TRAC, and B2M, respectively; wherein the gRNAs are the sequences represented by SEQ ID NOs: 4, 14, 24, and 25, respectively; or the sequences represented by SEQ ID NOs: 4, 15, 24, and 25, respectively; or the sequences represented by SEQ ID NOs: 4, 23, 24, and 25, respectively; or the sequences represented by SEQ ID NOs: 12, 14, 24, and 25, respectively; or the sequences represented by SEQ ID NOs: 12, 15, 24, and 25, respectively; or the sequences represented by SEQ ID NOs: 12, 23, 24, and 25, respectively; or the sequences represented by SEQ ID NOs: 13, 14, 24, and 25, respectively; or the sequences represented by SEQ ID NOs: 13, 15, 24, and 25, respectively; or the sequences represented by SEQ ID NOs: 13, 23, 24, and 25, respectively; or the sequences represented by SEQ ID NOs: 4, 14, 24, and 66, respectively; or the sequences represented by SEQ ID NOs: NO: 4, 15, 24, and 66; or the sequences represented by SEQ ID NO: 4, 23, 24, and 66, respectively; or the sequences represented by SEQ ID NOs: 12, 14, 24, and 66, respectively; or the sequences represented by SEQ ID NOs: 12, 15, 24, and 66, respectively; or the sequences represented by SEQ ID NOs: 12, 23, 24, and 66, respectively; or the sequences represented by SEQ ID NOs: 13, 14, 24, and 66, respectively; or the sequences represented by SEQ ID NOs: 13, 15, 24, and 66, respectively; or the sequences represented by SEQ ID NOs: 13, 23, 24, and 66, respectively.

After ICE assay analysis and next-generation sequencing (NGS) detection, the newly screened g-NKG2A-2 show higher target gene editing efficiency than g-NKG2A used in existing technologies; the off-target risk of whole-genome off-target effect detection was also much lower than that of g-NKG2A used in existing technologies.

The present application provides an engineered cell for reducing allogeneic immune rejection. The engineered cells have low or no endogenous HLA-II expression. The engineered cells have low or no endogenous NKG2A expression. The engineered cells have low or no endogenous B2M/HLA-II expression. The engineered cells have low or no endogenous B2M/TCR/HLA-II expression. The engineered cells have low or no endogenous B2M/TCR/HLA-II/NKG2A expression. The immune cells of the present application that have low or no endogenous HLA-II expression do not significantly activate allogeneic immune cells. Immune cells with low or no endogenous HLA-II expression can reduce allogeneic immune rejection reactions.

In one embodiment, CRISPR/Cas technology is used to construct an engineered cell. In one example, the gRNA used in the CRISPR/Cas9 technology for constructing the engineered cell comprises the sequences represented by SEQ ID NOs: 4, 14, 24, and 25; or comprises the sequences represented by SEQ ID NOs: 4, 15, 24, and 25; or comprises the sequences represented by SEQ ID NOs: 4, 23, 24, and 25; or comprises the sequences represented by SEQ ID NOs: 12, 14, 24, and 25; or comprises the sequences represented by SEQ ID NOs: 12, 15, 24, and 25; or comprises the sequences represented by SEQ ID NOs: 12, 23, 24, and 25; or comprises the sequences represented by SEQ ID NOs: 13, 14, 24, and 25; or comprises the sequences represented by SEQ ID NOs: 13, 15, 24, and 25; or comprises the sequences represented by SEQ ID NOs: 13, 23, 24, and 25; or comprises the sequences represented by SEQ ID NOs: 4 and/or 14; or comprises the sequences represented by SEQ ID NOs: 4 and/or 15; or comprises the sequences represented by SEQ ID NOs: 4 and/or 23; or comprises the sequences represented by SEQ ID NOs: 4, 24 and 25; or comprises the sequences represented by SEQ ID NOs: 12 and/or 14; or comprises the sequences represented by SEQ ID NOs: 12 and/or 15; or comprises the sequences represented by SEQ ID NOs: 12 and/or 23; or comprises the sequences represented by SEQ ID NOs: 12, 24 and 25; or comprises the sequences represented by SEQ ID NOs: 13 and/or 14; or comprises the sequences represented by SEQ ID NOs: 13 and/or 15; or comprises the sequences represented by SEQ ID NOs: 13 and/or 23; or comprises the sequences represented by SEQ ID NOs: 13, 24 and 25; or comprises the sequences represented by SEQ ID NOs: 14, 24 and 25; or comprises the sequences represented by SEQ ID NOs: 15, 24 and 25; comprises the sequences represented by SEQ ID NOs: 23, 24 and 25.

In one example, the gRNA used in the CRISPR/Cas9 technology for constructing the engineered cell comprises the sequences represented by SEQ ID NOs: 4, 24 and 25, and the efficiency of TCR/B2M/CIITA triple knockout is about 80%. In one example, the gRNA used in the CRISPR/Cas9 technology for constructing the engineered cells comprises the sequences represented by SEQ ID NOs: 12, 24 and 25, and the efficiency of TCR/B2M/CIITA triple knockout is about 80%.

In one example, the gRNA used in the CRISPR/Cas9 technology for constructing the engineered cells comprises the sequences represented by SEQ ID NOs: 4, 24 and 66, and the efficiency of TCR/B2M/CIITA triple knockout is about 80%. In one example, the gRNA used in the CRISPR/Cas9 technology for constructing the engineered cells comprises the sequences represented by SEQ ID NOs: 12, 24 and 66, and the efficiency of TCR/B2M/CIITA triple knockout is about 80%.

In one example, the gRNA used in the CRISPR/Cas9 technology for constructing the engineered cells comprises the sequences represented by SEQ ID NOs: 15, 24 and 25, and the efficiency of triple knockout of TCR/B2M/NKG2A is about 80%. In one example, the gRNA used in the CRISPR/Cas9 technology for constructing the engineered cells comprises the sequences represented by SEQ ID NOs: 15, 24 and 66, and the efficiency of triple knockout of TCR/B2M/NKG2A is about 80%.

In one example, the gRNA used in the CRISPR/Cas9 technology for constructing the engineered cells comprises the sequences represented by SEQ ID NOs: 4, 15, 24 and 25, and the efficiency of the TCR/B2M/CIITA/NKG2A quadruple knockout is about 80%. In one example, the gRNA used in the CRISPR/Cas9 technology for constructing the engineered cells comprises the sequences represented by SEQ ID NOs: 12, 15, 24 and 25, and the efficiency of the TCR/B2M/CIITA/NKG2A quadruple knockout is about 80%.

In one example, the gRNA used in the CRISPR/Cas9 technology for constructing the engineered cells comprises the sequences represented by SEQ ID NOs: 4, 15, 24 and 66, and the efficiency of the TCR/B2M/CIITA/NKG2A quadruple knockout is about 80%.

In one example, the gRNA used in the CRISPR/Cas9 technology for constructing the engineered cells comprises the sequences represented by SEQ ID NOs: 12, 15, 24 and 66, and the efficiency of the TCR/B2M/CIITA/NKG2A quadruple knockout is about 80%.

Due to the immunogenetic differences between the donor and the recipient (or host), during transplantation of an exogenous transplant from a donor, the exogenous transplant from the donor will be recognized and attacked by immune cells (such as NK cells) in the host, thereby inhibiting or eliminating the transplant from the donor and generating a host-versus-graft reaction (HVGR). For example, in allogeneic cell transplantation, the absence of HLA-I molecules in allogeneic cells may reduce the host CD8+-mediated cellular immune rejection. In one example, the present application provides immune cells with low or no endogenous HLA-II/B2M expression.

Graft-versus-host disease (GVHD) is caused by the diversity of TCRs of exogenous transplanted donor T lymphocytes, and their incompatibility with the host HLA molecules. Donor T lymphocytes recognize antigens on the host normal tissues, and then they amplify and release a series of cytokines, which greatly enhance the immune response of a transplant to host antigens, thereby attacking the host cells. In one example, the present application provides immune cells with low or no endogenous HLA-II/TCR expression. In one example, cells with low or no expression of endogenous TCR are prepared in the present application by knocking out the gene TRAC of α chain of the endogenous TCR through the CRISPR system.

Under repeated stimulation of target cells (e.g., tumor cells expressing target antigens), the endogenous NKG2A expression in donor immune cells of the exogenous transplant is upregulated, and the donor immune cells will be killed by the immune cells recognizing NKG2A in the composition of the present application. In addition, low or no expression of NKG2A may relieve the inhibitory effect of the immune cells themselves, thereby exerting stronger anti-tumor ability. In one example, the present application provides immune cells with low or no endogenous HLA-II/NKG2A expression.

In one example, the present application provides immune cells with low or no endogenous TCR/B2M/HLA-II expression. In one example, the present application provides immune cells with low or no endogenous TCR/B2M/HLA-II/NKG2A expression.

The above immune cells do not significantly activate allogeneic immune cells. The above-mentioned immune cells may reduce allogeneic immune rejection reactions.

In one example, the present application provides an immune cell expressing an exogenous receptor while having low or no endogenous TCR/B2M/HLA-II expression. In one example, the present application provides an immune cell expressing a CAR while having low or no endogenous TCR/B2M/HLA-II expression. In one example, the present application provides an immune cell expressing a CAR that recognizes an NKG2A polypeptide while having low or no endogenous TCR/B2M/HLA-II expression. The present application provides an immune cell expressing a CAR that recognizes an NKG2A polypeptide and tumor antigen while having low or no expression of endogenous TCR/B2M/HLA-II. In one example, the present application provides an immune cell expressing a CAR that recognizes a tumor antigen while having low or no expression of endogenous TCR/B2M/HLA-II. In one example, the present application provides an immune cell expressing a CAR that recognizes a BCMA polypeptide while having low or no endogenous TCR/B2M/HLA-II expression. The present application provides an immune cell expressing a CAR that recognizes NKG2A and BCMA polypeptides while having low or no endogenous TCR/B2M/HLA-II expression.

In one example, the present application provides an immune cell expressing an exogenous receptor while having low or no endogenous TCR/B2M/NKG2A expression. In one example, the present application provides an immune cell expressing a CAR while having low or no endogenous TCR/B2M/NKG2A expression. In one example, the present application provides an immune cell expressing a CAR that recognizes an NKG2A polypeptide while having low or no endogenous TCR/B2M/NKG2A expression. The present application provides an immune cell expressing a CAR that recognizes an NKG2A polypeptide and a tumor antigen while having low or no endogenous TCR/B2M/NKG2A expression. In one example, the present application provides an immune cell expressing a CAR that recognizes a tumor antigen while having low or no endogenous TCR/B2M/NKG2A expression. In one example, the present application provides an immune cell expressing a CAR that recognizes a BCMA polypeptide while having low or no endogenous TCR/B2M/NKG2A expression. The present application provides an immune cell expressing a CAR that recognizes an NKG2A and a BCMA polypeptide while having low or no endogenous TCR/B2M/NKG2A expression.

In one example, the present application provides an immune cell expressing an exogenous receptor while having low or no endogenous TCR/B2M/HLA-II/NKG2A expression. In one example, the present application provides an immune cell expressing a CAR while having low or no endogenous TCR/B2M/HLA-II/NKG2A expression. In one example, the present application provides an immune cell expressing a CAR that recognizes an NKG2A polypeptide while having low or no endogenous TCR/B2M/HLA-II/NKG2A expression. The present application provides an immune cell expressing a CAR that recognizes an NKG2A polypeptide and a tumor antigen while having low or no endogenous TCR/B2M/HLA-II/NKG2A expression. The present application provides an immune cell expressing a CAR that recognizes a tumor antigen while having low or no endogenous TCR/B2M/HLA-II/NKG2A expression. In one example, the present application provides an immune cell expressing a CAR that recognizes a BCMA polypeptide while having low or no endogenous TCR/B2M/HLA-II/NKG2A expression. The present application provides an immune cell expressing a CAR that recognizes an NKG2A and a BCMA polypeptide while having low or no endogenous TCR/B2M/HLA-II/NKG2A expression.

The above-mentioned immune cells that recognize tumor antigens, and/or immune cells that recognize NKG2A polypeptides and tumor antigens, can significantly kill tumor cells without significantly activating allogeneic immune cells. The immune cells that recognize tumor antigens, and/or the immune cells that recognize NKG2A polypeptides and tumor antigens, can significantly kill tumor cells with low allogeneic immune rejection reactions.

In one example, the present application provides a composition comprising: a first immune cell recognizing an NKG2A polypeptide while having low or no endogenous HLA-II expression, and/or a second immune cell recognizing a tumor and/or a pathogen antigen while having low or no endogenous HLA-II expression; optionally, the first and/or second immune cell has low or no endogenous B2M expression, low or no endogenous TCR expression, or low or no endogenous B2M and TCR expression. In one example, the present application provides a composition comprising: the first immune cell recognizing an NKG2A polypeptide while having low or no endogenous TCR/B2M/HLA-II expression, and/or a second immune cell recognizing a tumor and/or a pathogen antigen while having low or no endogenous TCR/B2M/HLA-II expression. In one example, the present application provides a composition comprising: a first immune cell recognizing an NKG2A polypeptide and a tumor antigen while having low expression or no expression of endogenous HLA-II, and/or a second immune cell recognizing a tumor antigen while having low expression or no expression of endogenous HLA-II; optionally, the first and/or second immune cell has low expression or no expression of endogenous B2M, low expression or no expression of endogenous TCR, or low expression or no expression of endogenous B2M and TCR. In one example, the present application provides a composition comprising: a first immune cell recognizing an NKG2A polypeptide and a tumor antigen while having low or no endogenous TCR/B2M/HLA-II/NKG2A expression, and/or a second immune cell recognizing a tumor antigen while having low or no endogenous TCR/B2M/HLA-II/NKG2A expression. The immune cells in the above composition do not significantly activate allogeneic immune cells, and the immune cells in the composition have longer survival time and/or expansion ability. The immune cells in the above composition have a low allogeneic immune rejection reaction; and the above composition comprising the first immune cell and the second immune cell exhibits a stronger cell killing effect in vivo and in vitro, as compared with the first immune cell or the second immune cell.

### Exogenous Receptors

The exogenous receptor in this application refers to a fusion molecule formed by connecting DNA fragments or corresponding cDNAs of proteins from different sources by gene recombination technology, which comprises an extracellular domain, a transmembrane domain and an intracellular domain, also known as a chimeric receptor, including but not limited to: a chimeric antigen receptor (CAR), a recombinant TCR receptor.

In one example, the exogenous receptor recognizes an NKG2A polypeptide. In one example, the exogenous receptor recognizes an NKG2A polypeptide and a BCMA polypeptide. In one example, the exogenous receptor recognizes a BCMA polypeptide. In one example, the exogenous receptor binds to the extracellular domain of the NKG2A polypeptide. In one example, the exogenous receptor binds to the extracellular domain of the BCMA polypeptide. In one example, the exogenous receptor binds to the extracellular domains of the NKG2A polypeptide and the BCMA polypeptide.

In one example, the exogenous receptor recognizes a pathogen antigen, e.g., for use in treating and/or preventing pathogen infection or other infectious disease, e.g., in an immunocompromised subject. Pathogen antigens include, but are not limited to antigens of: viruses, bacteria, fungi, protozoa, or parasites; viral antigens include, but are not limited to antigens of: cytomegalovirus (CMV), Epstein-Barr virus (EBV), human immunodeficiency virus (HIV), or influenza virus.

In one example, the exogenous receptor is a CAR. In one example, the CAR comprises an NKG2A antibody. In one example, CAR comprises an NKG2A antibody and an antibody recognizing a tumor antigen; wherein the antigen recognition domain of the CAR comprises an Fv specifically binding to an NKG2A polypeptide or a tumor antigen, respectively. In one example, the CAR comprises an NKG2A antibody and an antibody recognizing a pathogen antigen; wherin the antigen recognition domain of the CAR comprises an Fv specifically binding to an NKG2A polypeptide and a pathogen antigen, respectively. In one example, the CAR comprises an antibody fragment specifically binding to a tumor and/or a pathogen antigen.

In one example, the CAR comprises a tandem antibody fragment that specifically binds to an NKG2A polypeptide and a BCMA polypeptide; wherein the antigen recognition domain of the CAR comprises an Fv that specifically binds to an NKG2A polypeptide and a BCMA polypeptide, respectively.

In one aspect, the present application contemplates modification of the amino acid sequence for an original antibody or fragment (such as VH or VL) that produces a functionally equivalent molecule. For example, the anti-NKG2A or anti-BCMA binding domain, such as VH or VL, comprised in the modified CAR retains at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with the original anti-NKG2A or anti-BCMA binding domain, such as VH or VL.

The present application contemplates modification of the entire CAR molecule, for example, modification of one or more amino acid sequences of various domains of the CAR molecule, in order to generate a functionally equivalent molecule. The modified CAR molecule retains at least about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% identity with the original CAR molecule.

In one example, the antigen recognition binding domain of the CAR comprises a sequence represented by SEQ ID NOs: 27, 28, 29, 30, 31, 32, 33, 34, 35, 36, 37, 38, 39, 40, 41, 42, and/or 43. In one example, the antigen recognition binding domain of the CAR comprises a tandem antibody sequence represented by SEQ ID NOs: 46, 47, 48, 49, or 50. In one example, the CAR further comprises a sequence represented by SEQ ID NOs: 51, 52 or 52. In one example, the CAR comprises a sequences represented by 54, 55, 56, 57 and/or 58.

The present application provides an engineered cell that expresses an exogenous receptor and reduces allogeneic immune rejection. The engineered cells have low or no endogenous HLA-II expression. The engineered cells have low or no endogenous NKG2A expression. The engineered cells have low or no endogenous B2M/HLA-II expression. The engineered cells have low or no endogenous B2M/TCR/HLA-II expression. The engineered cells have low or no endogenous B2M/TCR/NKG2A expression. The engineered cells have low or no endogenous B2M/TCR/HLA-II/NKG2A expression. In one example, the engineered cells are constructed by the CRISPR/Cas technology.

### Engineered Cells

In one example, the engineered cells provided by the present application comprise: immune cells, neurons, epithelial cells, endothelial cells or stem cells. The stem cells comprise human pluripotent stem cells (including human induced pluripotent stem cells (iPSCs) and human embryonic stem cells). In one embodiment, the engineered cells comprise immune cells. In one example, the engineered cells are primary cells.

The immune cell may be a cell of lymphoid lineage. The lymphoid lineage (including B cells, T cells and natural killer (NK) cells) provides antibody production, regulation of the cellular immune system, detection of exogenous agents in the blood, detection of exogenous cells in the host, etc. Non-limiting examples of immune cells of the lymphoid lineage include: T cells, natural killer T (NKT) cells, and precursors thereof, including embryonic stem cells and pluripotent stem cells (e.g., stem cells that differentiate into lymphoid cells or pluripotent stem cells). T cells may be any type of T cells, including but not limited to: helper T cells, cytotoxic T cells, memory T cells (including central memory T cells, stem cell-like memory T cells (or stem-like memory T cells) and two effector memory T cells such as TEM cells and TEMRA cells), regulatory T cells (also known as suppressor T cells), natural killer T cells, mucosa-associated invariant T cells, γδ T cells or αβ T cells. The cytotoxic T cells (CTL or killer T cells) are T lymphocytes that are able to induce the death of infected somatic or tumor cells. The T cells of the subject may be engineered to express the exogenous receptors of the present application. In one example, the immune cell is B cell, monocyte, natural killer cell, basophil, eosinophil, neutrophil, dendritic cell, macrophage, regulatory T cell, helper T cell, cytotoxic T cell, other T cells, or a combination thereof.

In one example, the immune cells are T cells. In one example, the T cells may be CD4+ T cells and/or CD8+ T cells. In one example, the immune cells are CD3+ T cells. In one example, the cells of the present application comprise a cell population collected from PBMC cells after stimulation with CD3 magnetic beads. In one example, the cells of the present application are selected from: T cells, NK cells, cytotoxic T cells, NKT cells, macrophages, CIK cells, stem cells, and stem cell-derived immune cells or a combination thereof. In one example, the immune cells are selected from: autologous or allogeneic T cells, stem cell-derived T cells, primary T cells, or autologous T cells derived from human.

Immune cells (such as T cells) may be autologous, non-autologous (such as allogeneic), or derived in vitro from engineered progenitor or stem cells. The immune cells may be obtained from many sources, including peripheral blood mononuclear cells (PBMC), bone marrow, lymph node tissue, umbilical cord blood, thymus tissue, tissue from sites of infection, ascites, pleural effusions, spleen tissue, and tumors.

In certain aspects of the present application, T cells may be obtained from a blood sample collected from a subject by using any number of techniques known to those skilled in the art, such as Ficoll™ separation technology. In a preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, cells collected by apheresis may be washed to remove the plasma portion, and then the cells are placed in an appropriate buffer or culture medium for subsequent processing steps. Multiple rounds of selection may also be used in the context of the present application. In certain aspects, it may be desirable to perform a selection procedure, and use “unselected” cells during activation and expansion. The “unselected” cells may also be subjected to additional rounds of selection.

The composition of the present application can regulate the tumor microenvironment.

The source of unpurified CTLs may be any source known in the art, for example, bone marrow, fetal, neonatal or adult or other hematopoietic cell sources, such as fetal liver, peripheral blood or umbilical cord blood. Various techniques may be used to isolate cells. For example, negative selection may be used to initially deplete non-CTLs. mAbs are particularly useful for identifying markers associated with specific cell lineages, and/or differentiation stages of positive and negative selection.

Most of the terminally differentiated cells may be removed initially by relatively rough separation. For example, magnetic bead separation may be used initially to remove large numbers of irrelevant cells. In certain embodiments, at least about 80%, typically at least about 70%, of the total hematopoietic cells will be removed prior to isolating the cells.

Separation procedures include, but are not limited to: density gradient centrifugation; resetting; coupling to particles that alter cell density; magnetic separation by using antibody-coated magnetic beads; affinity chromatography; cytotoxic agents conjugated to or used in conjunction with mAbs, including but not limited to a complement and a cytotoxin; and panning with antibodies attached to a solid matrix (such as plates, chips, elutriation), or any other convenient technique.

Techniques for separation and analysis include, but are not limited to flow cytometry, which may have varying degrees of sophistication, for example, multiple color channels, low-angle and obtuse-angle light scatter detection channels, and impedance channels.

By using a dye associated with dead cells, such as propidium iodide (PI), cells may be selected among dead cells. In certain embodiments, cells are collected in a medium containing 2% fetal calf serum (FCS) or 0.2% bovine serum albumin (BSA), or any other suitable (such as sterile, isotonic) medium.

### Vector

Genetic modification of the engineered cells (such as T cells or NKT cells) may be accomplished by transducing a substantially homogeneous population of cells with a recombinant nucleic acid molecule. In one example, retroviral vectors (gamma-retrovirus or lentivirus) are used to introduce nucleic acid molecules into cells. For example, a polynucleotide encoding an exogenous receptor (e.g., CAR) may be cloned into a retroviral vector; in addition, a non-viral vector may also be used. Any suitable viral vector or non-viral delivery system may be used in the transduction. A CAR may be constructed with helper molecules (such as cytokines) in a single polycistronic expression cassette, multiple expression cassettes in a single vector or in multiple vectors. Examples of elements for generating polycistronic expression cassettes include, but are not limited to: various viral and non-viral internal ribosome entry sites (IRES, e.g., FGF-1 IRES, FGF-2 IRES, VEGF IRES, IGF-II IRES, NF-κB IRES, RUNX1 IRES, p53 IRES, Hepatitis A IRES, Hepatitis C IRES, Pestivirus IRES, Baculovirus IRES, Picornavirus IRES, Poliovirus IRES, and Encephalomyocarditis Virus IRES), and cleavable linkers (e.g., 2A peptides, e.g., P2A, T2A, E2A, and F2A peptides).

Other viral vectors that can be used include, for example, adenoviral, lentiviral and adeno-associated viral vectors, vaccinia virus, bovine papilloma virus, or herpes viruses, such as Epstein-Barr virus.

Non-viral methods may also be used for genetic modification of immune cells. For example, nucleic acid molecules may be introduced into immune cells by lipofection, asialomucoid-polylysine conjugation, or microinjection under surgical conditions. Other non-viral gene transfer methods include transfection in vitro by using liposomes, calcium phosphate, DEAE dextran, electroporation, and protoplast fusion. The nucleic acid molecule may also be first transferred into a cell type that may be cultured in vitro (e.g., autologous or allogeneic primary cells or their progeny), and then the cells (or their progeny) modified with the nucleic acid molecule are injected into the target tissue of the subject or injected systemically.

In one example, a CAR encoding a target antigen (exemplarily, NKG2A and/or BCMA tumor antigen) is introduced into a T cell to generate an immune cell in the composition of the present application, and optionally, a nucleic acid inhibitory molecule or a nucleic acid molecule of gRNA targeting endogenous TCR, B2M, CIITA and/or NKG2A is introduced into a T cell. In one example, an in vitro transcribed CAR nucleic acid molecule, a nucleic acid inhibitory molecule or gRNA targeting endogenous TCR, B2M, CIITA or NKG2A may be introduced into the cell through a transient transfection. An exemplary artificial DNA sequence is a sequence comprising portions of genes linked together to form an open reading frame encoding a fusion protein. The DNA portions that are joined together may be from a single organism or from multiple organisms.

The present application also provides nucleic acid molecules encoding one or more exogenous receptors (such as a CAR) described herein, and nucleic acid inhibitory molecules or nucleic acid molecules of gRNA targeting endogenous TCR, B2M, CIITA or NKG2A.

### Administration

The composition of the present application may be provided systemically or directly to a subject to induce and/or enhance an immune response to an antigen, and/or treat and/or prevent a tumor, a pathogen infection or an infectious disease. In one example, the composition of the present application is injected directly into the organ of interest (such as an organ affected by a tumor). Alternatively, the compositions of the present application may be delivered to the organ of interest indirectly, for example by administration into the circulatory system (e.g., vein, tumor vasculature). Expansion and differentiation agents may be provided prior to, concurrently with, or after administration of the composition, so as to increase the production of T cells, NKT cells, or CTL cells in vitro or in vivo.

The immune cells in the composition of the present application may comprise purified cell populations. Those skilled in the art may easily determine the percentage of immune cells of the present application in a population by various well-known methods, such as fluorescence activated cell sorting (FACS). In a population comprising the immune cells of the present application, suitable ranges of purity are from about 50% to about 55%, from about 5% to about 60%, and from about 65% to about 70%. In certain embodiments, the purity is from about 70% to about 75%, from about 75% to about 80%, or from about 80% to about 85%. In certain embodiments, the purity is from about 85% to about 90%, from about 90% to about 95%, or from about 95% to about 100%. Dosages may be readily adjusted by those skilled in the art (e.g., reduced purity may require an increased dose). The cells may be introduced by injection, catheter, or the like.

The composition of the present application may be a pharmaceutical composition comprising the immune cells of the present application or progenitor cells thereof and a pharmaceutically acceptable carrier. Administration may be autologous or allogeneic. For example, immune cells or progenitor cells may be obtained from one subject and administered to the same subject or to a different compatible subject. Peripheral blood-derived immune cells or their progeny (e.g., derived in vivo, ex vivo, or in vitro) may be administered by local injection, including catheter administration, systemic injection, local injection, intravenous injection, or parenteral administration. When the composition of the present application is administered, it may be formulated into a unit dose injectable form (solution, suspension, emulsion, etc.).

### Dosage Form

The composition of the present application may be conveniently provided in the form of sterile liquid preparations, such as isotonic aqueous solutions, suspensions, emulsions, dispersions or viscous compositions, which may be buffered to a selected pH. Liquid formulations are generally easier to prepare than gels, other viscous compositions, and solid compositions. Additionally, liquid compositions are somewhat more convenient to administer, especially by injection. On the other hand, viscous compositions may be formulated within an appropriate viscosity range to provide a longer contact time with a particular tissue. Liquid or viscous compositions may comprise a carrier, which may be a solvent or dispersion medium containing, for example, water, saline, phosphate-buffered saline, a polyol (e.g., glycerol, propylene glycol, liquid polyethylene glycol, and the like), and suitable mixtures thereof.

Various additives may be added to enhance the stability and sterility of the compositions, including antimicrobial preservatives, antioxidants, chelating agents, and buffers. Prevention of the action of microorganisms may be ensured by various antibacterial and antifungal agents, for example, parabens, chlorobutanol, phenol, sorbic acid, and the like. Prolonged absorption of the injectable pharmaceutical form may be brought about by the use of agents delaying absorption, for example, aluminum monostearate and gelatin. However, any vehicle, diluent or additive used will have to be compatible with the genetically modified immune cells or their progenitors.

The number of cells in the composition to be administered will vary depending on the subject being treated. More effective cells may be administered in smaller quantities. The exact determination of an effective dose may be determined according to the individual factors of each subject, including the size, age, sex, weight, and condition of the subject. Dosages may be readily determined by those skilled in the art from this disclosure and knowledge in the art.

The amounts of cells and optional additives, vehicles, and/or carriers in the compositions to be administered in the methods may be readily determined by those skilled in the art. Typically, any additives (other than the one or more active cells and/or the one or more reagents) are present in phosphate buffered saline in an amount of 0.001% to 50% (by weight) solution, and the active ingredient is present in the order of micrograms to milligrams, for example, from about 0.0001 wt % to about 5 wt %, from about 0.0001 wt % to about 1 wt %, from about 0.0001 wt % to about 0.05 wt %, or from about 0.001 wt % to about 20 wt %, from about 0.01 wt % to about 10 wt %, or from about 0.05 wt % to about 5 wt %. For any composition to be administered to animals or human, the following results may be determined: toxicity, for example, by determining the lethal dose (LD) and LD50 in an appropriate animal model, e.g., a rodent such as a mouse; the dosage of the composition, the concentration of the components therein, and the time for administration of the composition, so as to elicit an appropriate response.

### Treatment Method

The present application provides a method for inducing and/or increasing an immune response in a subject in need of the composition of the present application. The composition of the present application may be used to treat and/or prevent tumors in a subject. The composition of the present application may be used to prolong the survival of a subject suffering from a tumor. The composition of the present application may also be used to treat and/or prevent pathogen infection or other infectious diseases in human subjects such as those with compromised immune function. This method comprises administering an effective amount of a composition of the present application to achieve the desired effect, whether alleviating an existing condition or preventing a recurrence. For therapeutic purposes, the amount administered will be that amount effective to produce the desired effect. One or more administrations may be used to provide an effective amount. An effective amount may be provided as a bolus or by continuous infusion.

In one example, the composition of the present application may be used to treat a subject having tumor cells with low levels of surface antigens, for example due to recurrence of the disease, where the subject has received treatment that resulted in residual tumor cells. In certain embodiments, the tumor cells have a low density of the target molecule on the surface of the tumor cells.

In one example, the composition of the present application may be used to treat a subject with disease recurrence, wherein the subject has received an immune cell (e.g., T cell) comprising a single administration of a CAR, the CAR comprises an intracellular signaling domain comprising a co-stimulatory signaling domain (e.g., 4-1BBz CAR). In one example, the disease is a BCMA-positive tumor. The method comprises administering an effective amount of the composition of the present application to achieve the desired effect, alleviate the existing condition or prevent recurrence.

The compositions of the present application may be administered by any method known in the art, including but not limited to: intravenous, subcutaneous, intranodal, intratumoral, intrathecal, intrapleural, intraperitoneal, and direct administration to the thymus.

The present application provides a method for treating and/or preventing a tumor in a subject. The method may comprise administering an effective amount of the composition of the present application to a subject suffering from a tumor.

Non-limiting examples of tumors include: blood cancers (e.g., leukemia, lymphoma, and myeloma), ovarian cancer, breast cancer, bladder cancer, brain cancer, colon cancer, intestinal cancer, liver cancer, lung cancer, pancreatic cancer, prostate cancer, skin cancer, stomach cancer, glioblastoma, laryngeal cancer, melanoma, neuroblastoma, adenocarcinoma, glioma, soft tissue sarcomas, and various carcinomas (including prostate cancer and small cell lung cancer). Non-limiting examples of tumors include, but are not limited to: astrocytoma, fibrosarcoma, myxosarcoma, liposarcoma, oligodendroglioma, ependymomas, medulloblastoma, primitive neuroectodermal tumor (PNET), chondrosarcoma, osteogenic sarcoma, pancreatic ductal adenocarcinoma, small cell and large cell lung adenocarcinoma, chordoma, angiosarcoma, endotheliosarcoma, squamous cell carcinoma, bronchoalveolar carcinoma, epithelial adenocarcinoma and their liver metastases, lymphangiosarcomas, lymphangioendotheliosarcomas, hepatocarcinomas, bile duct carcinoma, synovioma, mesothelioma, Ewing's tumor, rhabdomyosarcoma, colon carcinoma, basal cell carcinoma, sweat gland carcinoma, papillary carcinoma, sebaceous gland carcinoma, thyroid cancer, cystadenocarcinoma, medullary carcinoma, bronchial carcinoma, renal cell carcinoma, cholangiole carcinoma, choriocarcinoma, seminoma, embryonal carcinoma, Wilms' tumor, testicular tumor, medulloblastoma, craniopharyngioma, ependymoma, pinealoma, hemangioblastoma, acoustic neuroma, oligodendroglioma, meningioma, neuroblastoma, retinoblastoma, leukemia, multiple myeloma, Waldenstrom's macroglobulinemia and heavy chain disease, breast tumors such as ductal and lobular adenocarcinoma, squamous and adenocarcinoma of the cervix, epithelial carcinoma of the uterus and ovary, adenocarcinoma of the prostate, transitional squamous cell carcinoma of the bladder, B and T cell lymphoma (nodular and diffuse) plasmacytoma, acute and chronic leukemia, malignant melanoma, soft tissue sarcoma and leiomyosarcoma. In certain embodiments, the tumor is selected from: blood cancer (e.g., leukemia, lymphoma, and myeloma), ovarian cancer, prostate cancer, breast cancer, bladder cancer, brain cancer, colon cancer, intestinal cancer, liver cancer, lung cancer, pancreatic cancer, prostate cancer, skin cancer, stomach cancer, glioblastoma, and laryngeal cancer. In one example, the composition of the present application may be used to treat and/or prevent solid tumors that are not suitable for conventional treatment measures or are refractory to recurrence, such as liver cancer, lung cancer, breast cancer, ovarian cancer, kidney cancer, thyroid cancer, gastric cancer, and colorectal cancer. In one embodiment, the tumor is a hematological tumor.

The therapeutic goal of the composition of the present application may comprise alleviating or reversing disease progression and/or alleviating side effects, or the therapeutic goal may comprise reducing or delaying the risk of recurrence.

The present application provides a method for treating and/or preventing pathogen infection (e.g., viral infection, bacterial infection, fungal infection, parasitic infection, or protozoan infection), for example, in an immunocompromised subject. The method may comprise administering an effective amount of a composition of the present application to a subject suffering from a pathogen infection. Exemplary viral infections that are amenable to treatment include, but are not limited to infection of: cytomegalovirus, Epstein-Barr virus, human immunodeficiency virus, and influenza virus.

The term “enhance” refers to allowing a subject or tumor cell to improve its ability to respond to a treatment disclosed herein. For example, an enhanced response may comprise a 5%, 10%, 15%, 20%, 25%, 30%, 35%, 40%, 45%, 50%, 55%, 60%, 65%, 70%, 75%, 80%, 85%, 90%, 95% or 98% or more increase in responsiveness. As used herein, the term “enhance” may also refers to increasing the number of subjects who respond to a treatment, such as immune cell therapy. For example, an enhanced response may refers to the total percentage of subjects that respond to treatment, wherein the percentage is 5%, 10%, 15%, 20%, 25%, 30%, 35%, 40%, 45%, 50%, 55%, 60%, 65%, 70%, 75%, 80%, 85%, 90%, 95% or 98% more.

In one example, the composition targets tumors that are positive for BCMA expression. In one example, the composition targets multiple myeloma.

### Kit

The present application provides a kit for inducing and/or enhancing an immune response and/or treating and/or preventing a tumor or pathogen infection in a subject. In one example, the kit comprises an effective amount of the composition of the present application and a pharmaceutical composition. In one example, the kit comprises a sterile container; such a container may be a box, ampoule, bottle, vial, tube, bag, pouch, blister pack, or other suitable container known in the art. Such containers may be made of plastic, glass, laminated paper, metal foil, or other material suitable for containing medications. In one example, the kit comprises a nucleic acid molecule encoding the CAR of the present application, which recognizes the target antigen in an expressible form and may be optionally contained in one or more vectors.

In one example, the composition and/or nucleic acid molecule of the present application is provided together with instructions for administering the composition or nucleic acid molecule to a subject suffering from a tumor, pathogen, or immune disease or at risk of developing a tumor, pathogen, or immune disease. The instructions generally include information regarding the use of the composition for treating and/or preventing tumors or pathogen infection. In one example, the instructions include at least one of the following: a description of the therapeutic agent; dosage and administration schedules for treating or preventing tumors, pathogen infections, or immune diseases, or symptoms thereof; precautions; warnings; indications; contraindications; medication information; adverse reactions; animal pharmacology; clinical studies; and/or references. These instructions may be printed directly on the container, or as a label affixed to the container, or provided within or with the container as a separate sheet, booklet, card or folder.

### Advantages of this Application

The BiTE or engineered cells secreting BiTE provided in the present application have a killing effect on NK cells, providing a new treatment method for anti-NK cell tumors. Furthermore, a method for increasing the durability and/or transplant survival rate of allogeneic immune cells in the presence of host immune cells is also provided herein.

This application comprises the CAR-T cells and preparation methods thereof disclosed in, for example, the following Chinese patent application publications: CN107058354A, CN107460201A, CN105194661A, CN105315375A, CN105713881A, CN106146666A, CN106519037A, CN106554414A, CN105331585A, CN10639759 3A, CN106467573A, CN104140974A, CN108884459A, CN107893052A, CN108866003A, CN108853144A, CN109385403A, CN109385400A, CN109468279A, CN109503715A, CN109908176 A, CN109880803A, CN110055275A, CN110123837A, CN110438082A, CN110468105A, and the following international patent application publications: WO2017186121A1, WO2018006882A1, WO2015172339A8, WO2018/018958A1, WO2014180306A1, WO2015197016A1, WO2016008405A1, WO2016086813A1, WO2016150400A1, WO2017032293A1, WO2017080377A1, WO2017186121A1, WO2018045811A1, WO2018108106A1, WO 2018/219299, WO2018/210279, WO2019/024933, WO2019/114751, WO2019/114762, WO2019/141270, WO2019/149279, WO2019/170147A1, WO 2019/210863, and WO2019/219029.

The present application is further described below in conjunction with specific Examples. It should be understood that these Examples are only used to illustrate the present application but not to limit the scope of the present application. The experimental methods in the following examples without specifying specific conditions are usually carried out according to conventional conditions described in Sambrook, J., et al., Molecular Cloning: A Laboratory Manual, Third Edition, Science Press (2002), or the conditions recommended by the manufacturer. All publications, patents, and patent applications mentioned in this specification are herein incorporated by reference to the same extent as if each individual publication, patent, or patent application was specifically and individually indicated to be incorporated by reference.

### Example 1: Preparation of Endogenous Gene Knockout Cells by CRISPR/Cas9 Technology

Conventional CRISPR/Cas9 technology was used to knock out endogenous genes in T cells. Briefly, human PBMCs were isolated in vitro, activating with anti-CD3/CD28 magnetic beads for 48 hours, and then transfecting with lentivirus expressing CAR (when preparing UCAR-T cells). After 96 hours, the following gene knockout operations were performed: Cas 9 enzyme (purchased from Kactus Biosystems) and gRNA targeting endogenous genes were mixed in a ratio of 1:4 to form an RNP complex, incubating at room temperature and then adding to T cells. The RNP complex was introduced into T cells by using a MaxCyte or Lonza electroporator to prepare T cells with endogenous gene knockout.

### Example 2: Screening of gRNA for Efficient Knockout of CIITA

Thirteen gRNAs targeting the CIITA gene were designed through the CRISPR/Cas9-gRNA design website. After the corresponding primers of gRNA were synthesized in vitro (purchased from GENEWIZ), gRNA was transcribed and amplified by using an in vitro gRNA transcription kit (purchased from Thermo Fisher).

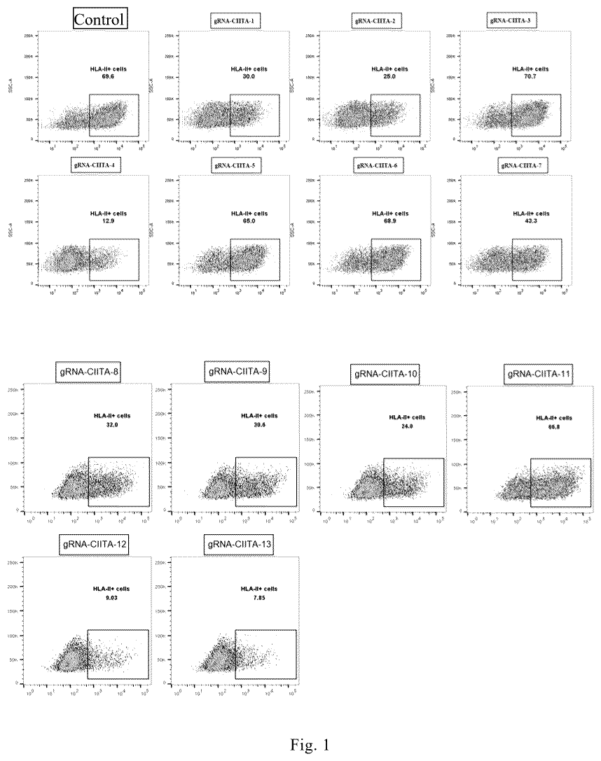

The method described in Example 1 was used to knock out endogenous CIITA in T cells. The knockout efficiency of CIITA was detected by flow cytometry using HLA-II antibody (purchased from BD Biosciences). The results of CIITA knockout efficiency under the concentration of 1 μM Cas9 are shown in FIG. 1 and Table 1. Under the condition of the concentration of 0.5 μM cas9 enzyme, the knockout efficiencies of g-CIITA-4, 12, and 13 are 70.9%, 67.5%, and 33.1%, respectively.

TABLE 1
Gene editing efficiency of gRNA sequences targeting
CIITA gene (cas9 = 1 μM)

| Sequence name | Sequence number | Gene editing efficiency |
|---|---|---|
| g-CIITA-1 | SEQ ID NO: 1 | 56.9% |
| g-CIITA-2 | SEQ ID NO: 2 | 64.1% |
| g-CIITA-3 | SEQ ID NO: 3 | 0 |
| g-CIITA-4 | SEQ ID NO: 4 | 81.5% |
| g-CIITA-5 | SEQ ID NO: 5 | 6.6% |
| g-CIITA-6 | SEQ ID NO: 6 | 1.0% |
| g-CIITA-7 | SEQ ID NO: 7 | 37.8% |
| g-CIITA-8 | SEQ ID NO: 8 | 54.0% |
| g-CIITA-9 | SEQ ID NO: 9 | 56.0% |
| g-CIITA-10 | SEQ ID NO: 10 | 65.5% |
| g-CIITA-11 | SEQ ID NO: 11 | 4.0% |
| g-CIITA-12 | SEQ ID NO: 12 | 87.0% |
| g-CIITA-13 | SEQ ID NO: 13 | 88.7% |

### Example 3: Screening of gRNA for Efficient Knockout of NKG2A

A total of 9 gRNAs targeting the NKG2A gene were designed through the CRISPR/Cas9-gRNA design website, and the sequences are shown in Table 2. After the corresponding primers of each gRNA were synthesized in vitro (purchased from GENEWIZ), the gRNA was transcribed and amplified by using an in vitro gRNA transcription kit (purchased from Thermo Fisher).

TABLE 3
gRNAs targeting NKG2A gene

| Sequence name | Sequence number |
|---|---|
| g-NKG2A-1 | SEQ ID NO: 14 |
| g-NKG2A-2 | SEQ ID NO: 15 |
| g-NKG2A-3 | SEQ ID NO: 16 |
| g-NKG2A-4 | SEQ ID NO: 17 |
| g-NKG2A-5 | SEQ ID NO: 18 |
| g-NKG2A-6 | SEQ ID NO: 19 |
| g-NKG2A-7 | SEQ ID NO: 20 |
| g-NKG2A-8 | SEQ ID NO: 21 |
| g-NKG2A-9 | SEQ ID NO: 22 |

Endogenous NKG2A knockout T cells were prepared by referring to Example 1. The genomic DNA in the T cells was extracted for PCR amplification, and the gene editing efficiency of each gRNA was obtained by ICE assay analysis after Sanger sequencing. The screening results show that the gene editing efficiencies of g-NKG2A-1, 2, and 8 are 19%, 72%, and 9%, respectively.

### Example 4: Testing the Off-Target Effects of the Newly Screened NKG2A gRNA Whole Genome

Bioinformatics analysis software was used to perform a whole genome off-target analysis of the newly screened NKG2A-gRNA. The results show that the off-target risk of gRNA NKG2A-2 is very low at the whole genome level.

### Example 5: Preparation of Endogenous TCR, B2M, NKG2A, and CIITA Knockout Cells

BCMA-CAR-T cells expressing BCMA-CAR (SEQ ID NO: 54), NKG2A-CAR-T cells expressing NKG2A-CAR (SEQ ID NO: 57), and BCMA-NKG2A-CAR-T cells expressing BCMA-NKG2A-CAR (SEQ ID NO: 58) were constructed respectively by conventional molecular biological methods in the art.

Referring to Example 1, T cells were subjected to double knockout of TCR/B2M genes to obtain T-BT KO cells, or T cells were subjected to triple knockout of TCR/B2M/CIITA genes to obtain T-BTC KO cells, or T cells were subjected to quadruple knockout of TCR/B2M/CIITA/NKG2A genes to obtain T-FKO cells. The cells were labeled with anti-CD3, anti-B2M, and anti-HLA-II antibodies, and the knockout of TCR, B2M, and CIITA was detected by flow cytometry, and the knockout of NKG2A was detected by gene sequencing. The efficiency of TCR/B2M double knockout is about 85% (that is, the proportion of T cells with double knockout of TCR and B2M to total T cells is about 85%). The efficiency of TCR/B2M/CIITA triple knockout is about 80% (that is, the proportion of T cells with triple knockout of TCR, B2M and CIITA to total T cells is about 80%), and the gRNA sequences used herein comprise a combination of SEQ ID NOs: 4, 24 and 25, or a combination of SEQ ID NOs: 4, 24 and 66. The efficiency of TCR/B2M/NKG2A triple knockout is about 80% (that is, the proportion of T cells with triple knockout of TCR, B2M and NKG2A to total T cells is about 80%), and the gRNA sequences used herein comprise a combination of SEQ ID NOs: 4, 24 and 25, or a combination of SEQ ID NOs: 4, 24 and 66. The efficiency of quadruple knockout of TCR/B2M/CIITA/NKG2A genes is about 80% (that is, the proportion of T cells with quadruple knockout of TCR, B2M, CIITA and NKG2A to total T cells is about 80%), and the gRNA sequences used herein comprise a combination of SEQ ID NOs: 4, 14, 24 and 25, or a combination of SEQ ID NOs: 4, 14, 24 and 66; the gRNA sequences used herein comprise a combination of SEQ ID NOs: 12, 14, 24 and 25, or a combination of SEQ ID NOs: 12, 14, 24 and 66.

BCMA CAR-T cells were subjected to TCR/B2M double knockout, followed by removal of TCR/B2M positive cells to obtain BCMA-UCAR-T. BCMA-NKG2A CAR-T cells were subjected to TCR/B2M/NKG2A triple knockout, followed by removal of TCR/B2M positive cells to obtain BCMA-NKG2A-UCAR-T-TKO cells. BCMA CAR-T, BCMA-NKG2A-CAR-T, and BCMA-NKG2A-CAR-T cells were subjected to TCR/B2M/CIITA/NKG2A quadruple knockout, followed by removal of TCR/B2M/HLA-II positive cells to obtain BCMA-UCAR-T-FKO, NKG2A-UCAR-T-FKO, and BCMA-NKG2A-UCAR-T-FKO cells. UTD cells with TCR, B2M, NKG2A or CIITA gene knockout and not transfected with a CAR were used as negative controls. Particularly, the gRNA sequences targeting CIITA, NKG2A, TCR, and B2M are SEQ ID NOs: 4, 23, 24, and 25, respectively.

The gRNA sequences targeting CIITA, NKG2A, TCR and B2M are SEQ ID NOs: 4, 15, 24 and 25 respectively, and they are used to prepare the BCMA-UCAR-T-FKO-2 with quadruple knockout of TCR/B2M/CIITA/NKG2A.

### Example 6: Knockout of Endogenous HLA-II in UCAR-T Cells can Reduce Allogeneic Immune Rejection

5×10⁵ BCMA-UCAR-T-TKO and BCMA-UCAR-T-FKO cells were taken at a ratio of 1:1 to co-culture with allogeneic (a donor different from that of the T cells for preparing endogenous CIITA knockout) CD4+ T cells (labeled with CFSE), and CD3 and CD40L (markers of CD4+ T cell activation) staining were performed on day 3 (D3) and day 7 (D7) respectively, to detect the expression of CFSE in CD3+CD4+ T cells.

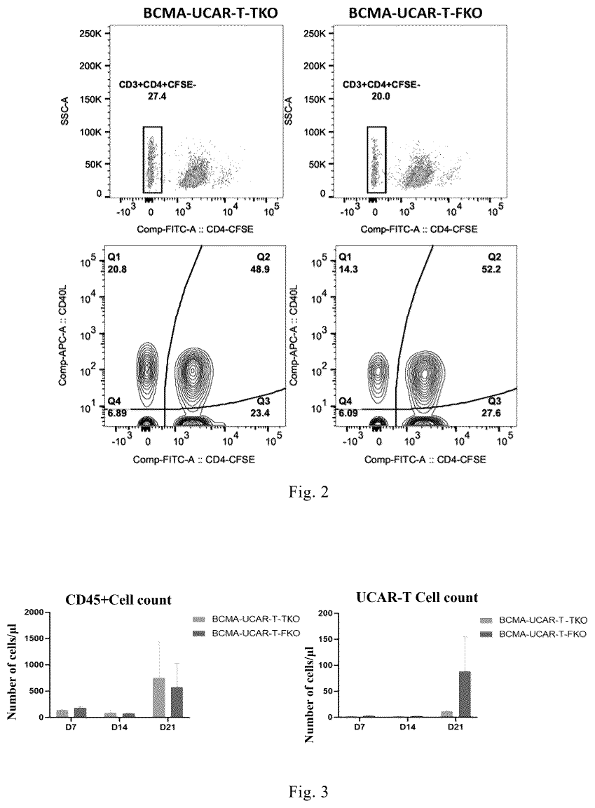

The results are shown in FIG. 2. On D7, the proportion of CFSE-negative cells of allogeneic CD4+ cells in the BCMA-UCAR-T-FKO group is significantly lower than that in the BCMA-UCAR-T-TKO group; and the proportion of CD40L+ in the CFSE-negative population in the BCMA-UCAR-T-FKO group is also lower than that in the BCMA-UCAR-T-TKO cell group. This suggests that T cells with endogenous CIITA knockout can reduce the activation of allogeneic CD4 immune cells.

The effect of endogenous CIITA knockout on T cell immune rejection response in vivo was further detected. NPG mice were divided into two groups. On day 1 (D1), 1×10⁶ BCMA-UCAR-T-TKO cells and BCMA-UCAR-T-FKO cells were injected respectively. 24 hours after injection, allogeneic PBMC cells activated and expanded in vitro were injected into each mouse at a dose of 6×10⁶ cells. After injection, blood samples were taken from mice at day 7 (D7) and day 14 (D14), and the number of total human cells (labeled with CD45 antibody) and the number of UCAR-T cells (labeled with CD45+CD3−) infused were detected by flow cytometry to calculate the survival of T cells. On day 15 (D15), 5×10⁶ BCMA-UCAR-T-TKO and BCMA-UCAR-T-FKO cells were injected again, and then the survival of UCAR-T cells was detected by flow cytometry on day 21 (D21).

The results are shown in FIG. 3. The counting results on D21 show that the total number of human cells (CD45+ cells) in the two groups of mice is comparable, while the number of UCAR-T (CD45+CD3−) cells in the BCMA-UCAR-T-FKO group is significantly higher than that in the BCMA-UCAR-T-TKO group, indicating that knocking out endogenous CIITA in UCAR-T cells can contribute to reducing the allogeneic immune rejection.

### Example 7: Anti-Tumor Effect of UCAR-T Cells with Endogenous CIITA Knockout

BCMA-UCAR-T-TKO and BCMA-UCAR-T-FKO were respectively prepared as effector cells, UTD cells were used as negative control; and RPMI-8226 multiple myeloma cells were used as target cells. The cells were incubated for 18 hours at effector-target ratios of 3:1, 1:1, and 1:3, respectively; and after centrifugation, LDH release was detected (purchased from Roche) to calculate the tumor cell lysis efficiency.

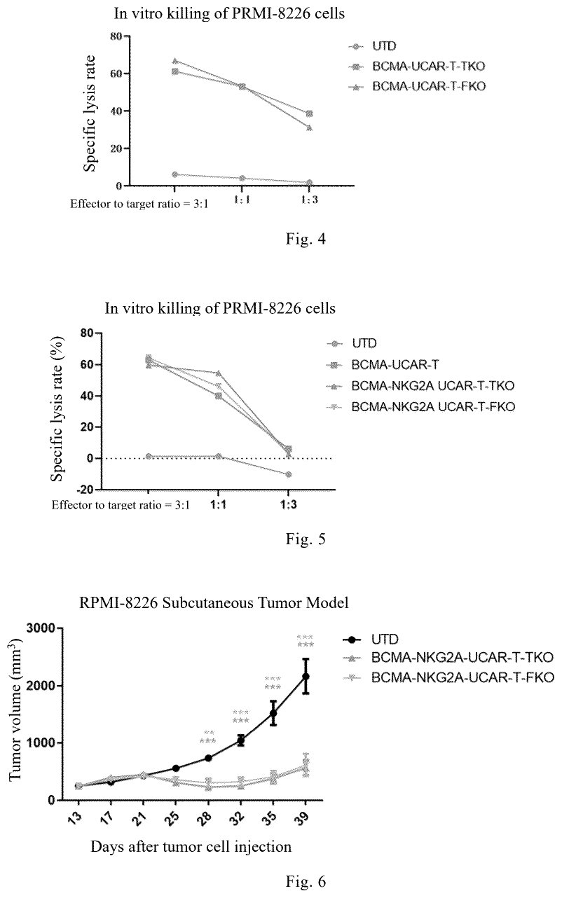

The results are shown in FIG. 4, and UCAR-T cells recognizing tumor antigens and having endogenous TCR/B2M/CIITA/NKG2A knockout, can kill tumor cells in vitro.

### Example 8: Anti-tumor Effect of Tandem UCAR-T Cells with Endogenous CIITA Knockout

### 1. In Vitro Anti-Tumor Experiment

BCMA-UCAR-T, BCMA-NKG2A-UCAR-T-TKO, and BCMA-NKG2A-UCAR-T-FKO as effector cells, UTD cells as negative control, and RPMI-8226 as target cells were incubated for 18 hours at effector-target ratios of 3:1, 1:1, and 1:3, respectively. After centrifugation, LDH release was detected (purchased from Roche) to calculate the tumor cell lysis efficiency.

The results are shown in FIG. 5, and the tandem UCAR-T cells recognizing NKG2A polypeptides and tumor antigens and having endogenous TCR/B2M/CIITA/NKG2A knockout, can kill tumor cells in vitro.

### 2. In Vivo Anti-Tumor Experiment

5×10⁶ RPMI-8226 cells were subcutaneously inoculated into NPG immunodeficient mice. 12-14 days after inoculation, the average tumor volume was about 200-250 mm³, and the mice were divided into three groups. 1×10⁶ UTD, BCMA-NKG2A-UCAR-T-TKO, and BCMA-NKG2A-UCAR-T-FKO cells were injected by tail vein. After injection, body weight was measured twice a week, and the long and short diameters of the tumor were measured with a vernier caliper and recorded to calculate the tumor volumes. The tumor growth curve was generated according to the tumor volumes, and the differences in tumor growth curves among the groups were compared (tumor volume: V=1/2× long diameter× short diameter²).

The results are shown in FIG. 6, and tandem UCAR-T cells recognizing NKG2A polypeptides and tumor antigens and having endogenous TCR/B2M/CIITA/NKG2A knockout, can exert anti-tumor effects in vivo.

### Example 9: Synergistic In Vivo and In Vitro Anti-Tumor Effects of UCAR-T Cells with Endogenous CIITA Knockout in the Presence of NK Cells

Primary NK cells were isolated from peripheral blood mononuclear cells by using an NK cell isolation kit (purchased from Miltenyi Biotec), and then expanded and cultured in vitro for 14 days by using NK cell culture medium containing IL-2.

1. In the Presence of NK Cells, UCAR-T Cells Recognizing NK Cell Markers and Having Endogenous CIITA Knockout, Promote the Survival and/or Expansion of UCAR-T Cells In Vitro.

The multiple myeloma MM.1S-GFP cells as target cells; primary cultured NK cells as Effector cell 1; and UCAR-T cells as Effector cell 2.

The groups are as follows:

Control groups: MM.1S alone group (negative control group, denoted as MM.1S-GFP), MM.1S+BCMA UCAR-T-FKO+UTD-FKO (denoted as +UTD-FKO), MM.1S+BCMA UCAR-T-FKO+UTD-FKO+NK (denoted as +UTD-FKO+NK);

NKG2A groups: MM.1S+BCMA UCAR-T-FKO+NKG2A UCAR-T-FKO (denoted as NKG2A UCAR-T-FKO), MM.1S+BCMA UCAR-T-FKO+NKG2A UCAR-T-FKO+NK (denoted as +NKG2A UCAR-T-FKO+NK).

Specific process: 3×10⁴ MM.1S-GFP were inoculated into a 96-well plate at two ratios of target cells:BCMA UCAR-T-FKO cells:NKG2A UCAR-T-FKO cells (or UTD-FKO cells): primary NK cells=2:2:2:1 and 2:2:2:2. After 5 days of co-culture, the proportion of CD45/HLA-ABC cells was detected by flow staining, and absolute cell quantification was performed. GFP positivity represents tumor cells, CD45+HLA-ABC+ cells represent NK cells, and CD45+HLA-ABC-cells represent UCAR-T cells.

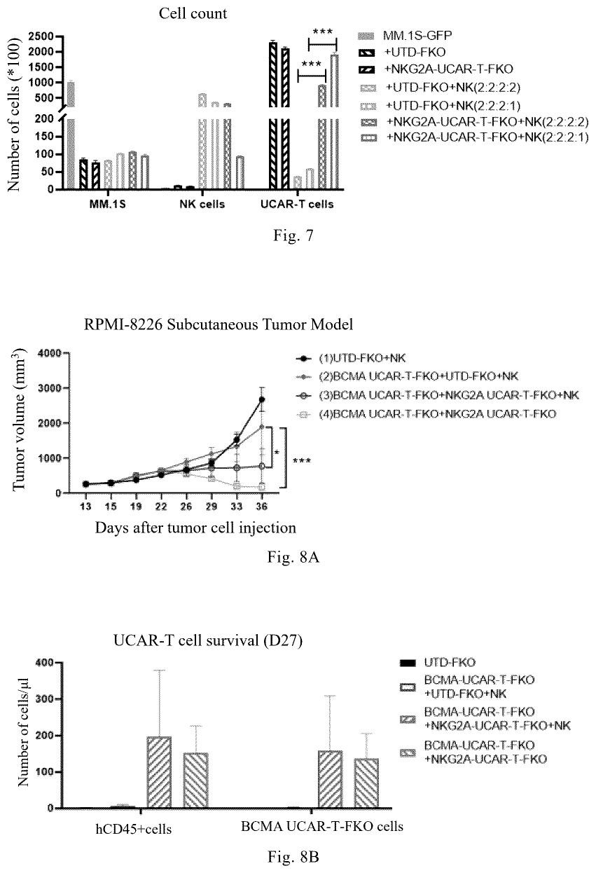

The results are shown in FIG. 7. In the presence of NK cells, UCAR-T cells recognizing an NKG2A and having endogenous TCR/B2M/CIITA/NKG2A knockout, can not only promote the in vitro survival and/or expansion of UCAR-T cells in the composition, but also exert a synergistic anti-tumor effect.

2. In the Presence of NK Cells, NKG2A-UCAR-T Cells with Endogenous CIITA Knockout Promote the Anti-Tumor Effect of UCAR-T Cells In Vivo

5×10⁶ RPMI-8226 cells were subcutaneously inoculated into NPG mice. 13 days after inoculation, the average tumor volume was about 250 mm³. The mice were divided into 4 groups as shown in FIG. 8, with 5 mice in each group. After grouping, 1×10⁶ BCMA UCAR-T-FKO cells and 1×10⁶ NKG2A UCAR-T-FKO or UTD cells were injected by tail vein. On D13, D15, D18, D20, and D22, 1×10⁶ NK cells were injected into the mice in the above groups by tail vein, for a total of 5 times. The tumor growth curve was generated according to the method described in Example 8, and the content of BCMA UCAR-T-FKO cells in the peripheral blood of mice was detected 14 days after UCAR-T cell injection. The results show that in the presence of NK cells, UCAR-T cells recognizing an NKG2A and having endogenous TCR/B2M/CIITA/NKG2A knockout, can promote the anti-tumor activity of UCAR-T cells in vivo (FIG. 8A); and can promote the proliferation and survival of UCAR-T cells in vivo (FIG. 8B).

### Example 10: Construction of BiTE Targeting NK Cells

BiTEs targeting NKG2A: A1-BiTE (SEQ ID NO: 60), A2-BITE (SEQ ID NO: 61), A3-BiTE (SEQ ID NO: 62), NKG2A-CD3 (SEQ ID NO: 59); and BiTE targeting NKP46: NKP46-CD3 (SEQ ID NO: 63) were constructed by conventional molecular biology techniques. The above BiTEs were constructed into the viral packaging plasmid PRRLsin by the conventional molecular cloning technique, and the green fluorescent protein (GFP) was co-expressed after the BiTE fragment for tracing T cells expressing BiTE. In Examples 12, 14, and 15, the BiTE-expressing lentivirus was transfected into T cells for experiments; and in Examples 13 and 16, the supernatant of T cells expressing BiTE was collected for experiments.

### Example 11: Binding Property of Bispecific Antibodies Targeting NKG2A

Flow cytometry analysis show that the purified A1-BiTE, A2-BiTE, A3-BiTE, NKG2A-CD3, and NKP46-CD3 can specifically bind to CD3-positive Jurkat cells, and NKG2A-positive NK cells or NK92 cells.

### Example 12: NK Cells were Killed by BiTE-T Cells

T cells were transfected with lentivirus comprising A1-BiTE, A2-BiTE, A3-BiTE, NKG2A-CD3, and NKP46-CD3 respectively by conventional molecular biology techniques, to obtain A1-BiTE-T, A2-BITE-T, A3-BITE-T, NKG2A-CD3T, and NKP46-CD3 T cells, and T cells that were not transfected with the virus (UTD) were used as a control.

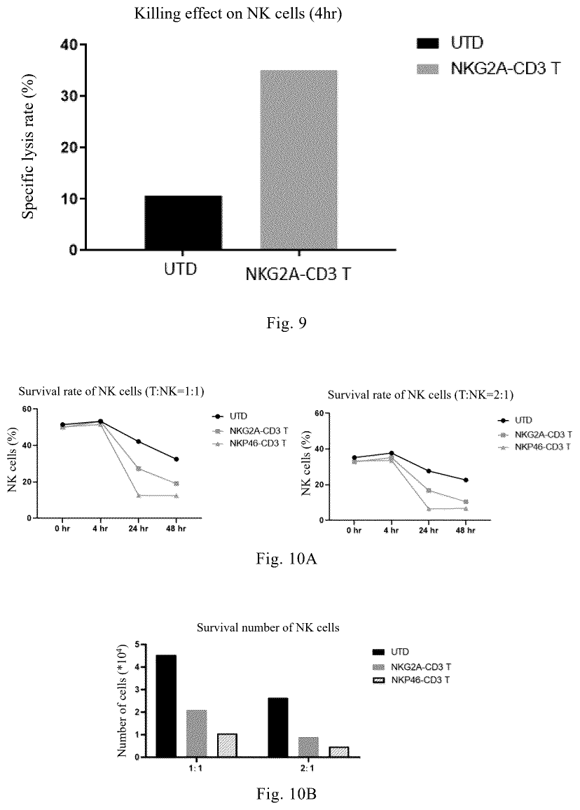

NKG2A-CD3 T cells were co-cultured with in vitro expanded NK cells at a ratio of 1:1. After 4 hours, the killing efficiency of T cells on NK cells was detected by using an LDH kit (purchased from Promega). The results are as shown in FIG. 9, and T cells expressing the NKG2A-CD3 bifunctional antibody can effectively lyse NK cells in vitro.

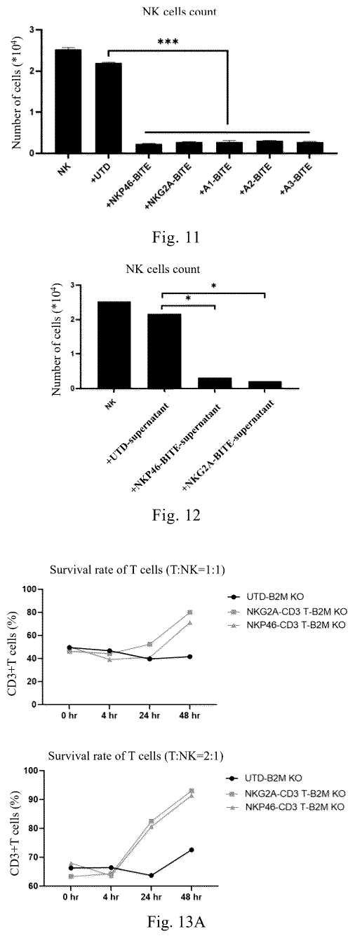

The above-mentioned NKG2A-CD3 T cells and NKP46-CD3 T cells were co-cultured with the in vitro expanded NK cells at a ratio of 1:1 or 2:1, then counting at 0, 4, 24 and 48 hours. The results show that the proportion of NK cells in the bifunctional antibody expression group is significantly lower than that in the UTD group (FIG. 10A); when counting after 48 hours of culture, the number of NK cells in the bifunctional antibody expression group is significantly lower than that in the UTD group (FIG. 10B). BiTE-T cells and NK cells were inoculated into a 96-well plate at a ratio of 1:1, then counting after 48 hours of culture. The results are as shown in FIG. 11, the number of NK cells in the BiTE-T group is significantly lower than that in the UTD group (P<0.001).

### Example 13: The Killing Effect of Bifunctional Antibodies on NK Cells

T cells expressing NKG2A-BiTE and NKP46-BiTE were cultured in basal medium (RPMI-1640+10% FBS) for 48 hours, and then the culture supernatants were collected respectively. The supernatant of UTD cells was used as a control. NK cells and UTD cells were inoculated into a 96-well plate at a ratio of 1:1, and the culture medium was replaced with the above-collected culture supernatant, and the cells were counted after 48 hours of culture.

The results are as shown in FIG. 12. The number of NK cells in the culture supernatant group supplemented with NKG2A-BiTE and NKP46-BiTE is significantly lower than that in the UTD supernatant group (P<0.05).

### Example 14: T Cells Expressing NKG2A-CD3 or NKP46-CD3 Bifunctional Antibodies can Effectively Resist NK Cell Killing Under the Condition of B2M Deficiency

B2M in BiTE-T cells was knocked out by the CRISPR/Cas9 technology to obtain BiTE-T-B2M KO cells with B2M knockout. The control cells, UTD-B2M KO, are T cells in which B2M is knocked out but BiTE is not expressed.

BiTE-T-B2M KO cells were co-cultured with in vitro expanded NK cells at a ratio of 1:1 or 2:1, then counting at 0, 4, 24, and 48 hours.

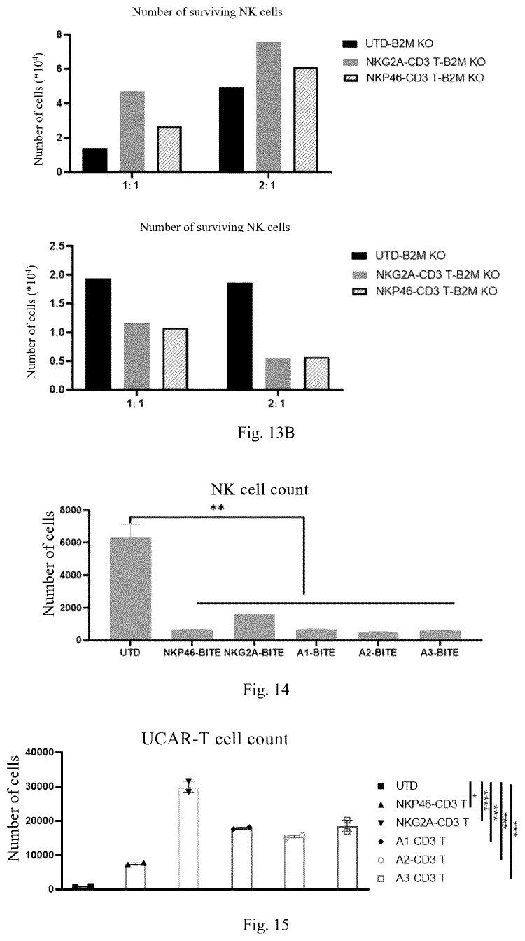

The results show that the proportion of T cells expressing bifunctional antibodies gradually increases over time, and the upward trend is significantly better than that of the control group (FIG. 13A). The counting results after 48 hours culture show that compared with the control group, the number of NK cells in the bifunctional antibody expression group is decreased, and the T cell number is increased (FIG. 13B), indicating that in the absence of B2M, T cells expressing bifunctional antibodies can effectively resist the attack of NK cells and have better survival ability.

### Example 15: T Cells Expressing Bifunctional Antibodies can Promote the Survival of UCAR-T Cells in the Presence of NK Cells

MM.1S cells, primary NK cells, UCAR-T cells, and BiTE-T cells were inoculated into a 96-well plate at a ratio of 1:1:1:1. After 5 days of co-culture, flow staining and absolute cell counting were performed by using the three antibodies (i.e., anti-CD45/anti-HLA-ABC/anti-CD3) to detect the number of tumor cells, NK cells, and UCAR-T cells, respectively.

The detection results are as shown in FIG. 14. Compared with the UTD group, the number of NK cells expressing BiTE in each group is significantly decreased (P<0.01), indicating that BiTE can effectively inhibit NK cells; in addition, compared with the UTD group, the number of BCMA-UCAR-T-FKO-2 cells expressing BiTE in each group is significantly increased (FIG. 15, P<0.01), indicating that BiTE targeting NK cells can promote the survival and expansion of UCAR-T cells.

### Example 17: Bifunctional Antibodies can Reduce the Immune Rejection of NK Cells to UCAR-T Cells

The supernatant of the culture medium expressing NKG2A-BiTE and NKP46-BiTE was used to verify their functions. MM.1S cells, NK cells, BCMA-UCAR-T-FKO-2 cells, and UTD cells were inoculated into a 96-well plate at a ratio of 1:1:1:1, and then the culture medium supernatant containing BiTE-T cells was added respectively. After 5 days of culture, flow staining and absolute cell counting were performed by using the three antibodies (i.e., anti-CD45/anti-HLA-ABC/anti-CD3) to detect the number of tumor cells, NK cells, and UCAR-T cells, respectively.

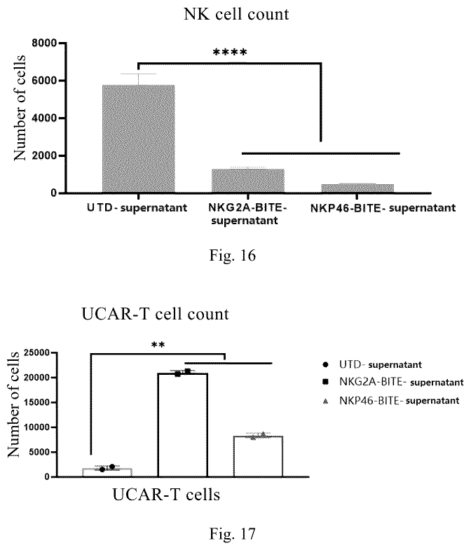

The test results are as shown in FIG. 16. Compared with the group with addition of UTD cell supernatant, the addition of supernatant containing NKG2A-BiTE and NKP46-BiTE cells can significantly inhibit the number of NK cells and promote the number of UCAR-T cells (FIG. 17). In summary, in the presence of NK cells, BiTE targeting NK cells can reduce the immune rejection of NK cells to UCAR-T cells and promote the survival and expansion of UCAR-T cells.

The examples described herein comprise the example as any single example or in combination with any other example or part thereof. In addition, it should be understood that after reading the above teachings of this application, those skilled in the art may make various changes or modifications to this application, and these equivalent forms also fall within the scope defined by the attached claims herein.

### Sequence Information

| No. | name | Sequences |
|---|---|---|
| 1 | g-CIITA-1 | GCTGAACTGGTCGCAGTTGA |
| 2 | g-CIITA-2 | GATATTGGCATAAGCCTCCC |
| 3 | g-CIITA-3 | TCAACTGCGACCAGTTCAGC |
| 4 | g-CIITA-4 | GAGAAGACAAAGTCGTACTG |
| 5 | g-CIITA-5 | GCCATTGCTTGAACCGTCCG |
| 6 | g-CIITA-6 | GCCACAGCCCTACTTTGTGC |
| 7 | g-CIITA-7 | GGTCCATCTGGTCATAGAAG |
| 8 | g-CIITA-8 | GAGATTCAGGCAGCTCAACG |
| 9 | g-CIITA-9 | GGACAGCTCAAATAGGGCGT |
| 10 | g-CIITA-10 | CATCAAAGTACCCTACAGG |
| 11 | g-CIITA-11 | AGAGTCCCGTGAGCGTGGA |
| 12 | g-CIITA-12 | GTCTAGGATGAGCAGAACG |
| 13 | g-CIITA-13 | CACGAGTGATTGCTGTGCT |
| 14 | g-NKG2A-1 | AGATAAGACAGATAATTCCC |
| 15 | g-NKG2A-2 | TGAACAGGAAATAACCTATG |
| 16 | g-NKG2A-3 | AAACCATTCATTGTCACCCA |
| 17 | g-NKG2A-4 | ATATTATTGAAGATCCACAC |
| 18 | g-NKG2A-5 | ACTGCAGAGATGGATAACCA |
| 19 | g-NKG2A-6 | GACAAAACCTATCACTGCAA |
| 20 | g-NKG2A-7 | CTGAATACAAGAACTCAGAA |
| 21 | g-NKG2A-8 | ATGGGTGACAATGAATGGTT |
| 22 | g-NKG2A-9 | TCCAACAGTTGTTACTACAT |
| 23 | g-NKG2A | GGTCTGAGTAGATTACTCCT |
| 24 | g-TRAC-1 | AGAGTCTCTCAGCTGGTACA |
| 25 | g-B2M-1 | GAGTAGCGCGAGCACAGCTA |
| 26 | crRNA/ | GTTTTAGAGCTAGAAATAGCAAGTTAAAATAAGGCTAGTCCGTTATCAACTTGAAAAA |
| | tracrRNA | GTGGCACCGAGTCGGTGCTTTT |
| 27 | BCMA | EVQLLESGGGLVQPGGSLRLSCAASGFTFGGNAMSWVRQAPGKGLEWVSAISGNGGST |
| | antibody1- | FYADSVKGRFTISRDNSKNTLYLQMNSLRAEDTAVYYCAKVRPFWGTFDYWGQGTLVT |
| | VH | VSS |
| 28 | BCMA | EIVLTQSPGTLSLSPGERATLSCRASQSVSSSYLAWYQQKPGQAPRLLIYGASSRATGIPD |
| | antibody1- | RFSGSGSGTDFTLTISRLEPEDFAVYYCQQYFNPPEYTFGQGTKVEIKR |
| | VL | |
| 29 | BCMA | evqllesggglvqpggslrlscaasgftfggnamswvrqapgkglewvsaisgnggstfyadsvkgrftisrdnskntlyl |
| | antibody1- | qmnslraedtavyycakvrpfwgtfdywgqgtlvtvssggggsggggsggggseivltqspgtlslspgeratlscrasqs |
| | scFv | vsssylawyqqkpgqaprlliygassratgipdrfsgsgsgtdftltisrlepedfavyycqqyfnppeytfgqgtkveik |
| | | r |
| 30 | BCMA | Evqllesggglvqpggslrlscaasgftfssyamswvrqapgkglewvsaisgsggstyyadsvkgrftisrdnskntlyl |
| | antibody2- | qmnslraedtavyycarypylafDywgqgtlvtvssggggsggggsggggseivltqspgtlslspgeratlscrasqsvs |
| | scFv | ssylawyqqkpgqaprlliygassratgipdrfsgsgsgtdftltisrlepedfavyycqqygyppsytfgqgtkveikr |
| 31 | BCMA | Evqllesggglvqpggslrlscaasgftfssyamswvrqapgkglewvsaisgsggstyyadsvkgrftisrdnskntlyl |
| | antibody3- | qmnslraedtavyycaklsgdaAmdywgqgtlvtvssggggsggggsggggseivltqspgtlslspgeratlscrasqsv |
| | scFv | sssylawyqqkpgqaprlliygassratgipdrfsgsgsgtdftltisrlepedfavyycqqygypprytfgqgtkveikr |
| 32 | BCMA | Evqllesggglvqpggslrlscaasgftfssyamswvrqapgkglewvsaisgsggstyyadsvkgrftisrdnskntlyl |
| | antibody4- | qmnslraedtavyycakvrpfwgtfdywgqgtlvtvssggggsggggsggggseivltqspgtlslspgeratlscrasqs |
| | scFv | vsssylawyqqkpgqaprlliygassratgipdrfsgsgsgtdftltisrlepedfavyycqqyfnppeytfgqgtkveik |
| | | r |
| 33 | BCMA | evqllesggglvqpggslrlscaasgftfrsyamswvrqapgkglewvsaisggggntfyadsvkgrftisrdnskntlyl |
| | antibody5- | qmnslraedtavyycakvrpfwgtfdywgqgtlvtvssggggsggggsggggseivltqspgtlslspgeratlscrasqs |
| | scFv | vsssylawyqqkpgqaprlliygassratgipdrfsgsgsgtdftltisrlepedfavyycqqyfnppeytfgqgtkveik |
| | | r |
| 34 | NKG2A | QVQLVQSGAEVKKPGASVKVSCKASGYTFTSYWMNWVRQAPGQGLEWMGRIDPYDS |
| | antibody 1-VH | ETHYAQKLQGRVTMTTDTSTSTAYMELRSLRSDDTAVYYCARGGYDFDVGTLYWFFDV |
| | | WGQGTTVTVSS |
| 35 | NKG2A | DIQMTQSPSSLSASVGDRVTITCRASENIYSYLAWYQQKPGKAPKLLIYNAKTLAEGVPS |
| | antibody 1-VL | RFSGSGSGTDFTLTISSLQPEDFATYYCQHHYGTPRTFGGGTKVEIK |
| 36 | NKG2A | EVQLLESGGGLVQPGGSLRLSCAASGFTFNRFYMSWVRQAPGKGLEWVSAITGWGGST |
| | antibody (A1)- | YYADSVKGRFTISRDNSKNTLYLQMNSLRAEDTAVYYCAKGYDGFDYWGQGTLVTVSS |
| | VH | |
| 37 | NKG2A | DIQMTQSPSTLSASVGDRVTITCRASQSISSWLAWYQQKPGKAPKLLIYDASSLESGVPSR |
| | antibody (A1)- | FSGSGSGTEFTLTISSLQPDDFATYYCQQYDSYVSTFGQGTKVEIKR |
| | VL | |
| 38 | NKG2A | EVQLLESGGGLVQPGGSLRLSCAASGFTFGRVHMSWVRQAPGKGLEWVSAISAGGGST |
| | antibody (A2)- | YYADSVKGRFTISRDNSKNTLYLQMNSLRAEDTAVYYCAKGYDGFDYWGQGTLVTVSS |
| | VH | |
| 39 | NKG2A | DIQMTQSPSTLSASVGDRVTITCRASQSISSWLAWYQQKPGKAPKLLIYDASSLESGVPSR |
| | antibody (A2)- | FSGSGSGTEFTLTISSLQPDDFATYYCQQYDSYVSTFGQGTKVEIKR |
| | VL | |
| 40 | NKG2A | EVQLLESGGGLVQPGGSLRLSCAASGFTFRNFHVSWVRQAPGKGLEWVSAINGPVGST |
| | antibody (A3)- | YYADSVKGRFTISRDNSKNTLYLQMNSLRAEDTAVYYCAKGYDGFDYWGQGTLVTVSS |
| | VH | |
| 41 | NKG2A | DIQMTQSPSTLSASVGDRVTITCRASQSISSWLAWYQQKPGKAPKLLIYDASSLESGVPSR |
| | antibody (A3)- | FSGSGSGTEFTLTISSLQPDDFATYYCQQYDSYVSTFGQGTKVEIKR |
| | VL | |
| 42 | NKP46 | QVQLQQPGSVLVRPGASVKLSCKASGYTFTSSWMHWAKQRPGQGLEWIGHIHPNSGIS |
| | antibody-VH | NYNEKFKGKATLTVDTSSSTAYVDLSSLTSEDSAVYYCSRGGRFDDWGAGTTVTVSS |
| 43 | NKP46 | DIQMTQSPSSLSASLGERVSLTCRASQDIGSSLNWLQQEPDGTIKRLIYATSRLDSGVPKRF |
| | antibody-VL | SGSRSGSDYSLTISSLESEDFVDYYCLQYASSPWTFGGGTKLEIKR |
| 44 | CD3 antibody- | DIKLQQSGAELARPGASVKMSCKTSGYTFTRYTMHWVKQRPGQGLEWIGYINPSRGYT |
| | VH | NYNQKFKDKATLTTDKSSSTAYMQLSSLTSEDSAVYYCARYYDDHYCLDYWGQGTTLT |
| | | VSS |
| 45 | CD3 antibody- | DIQLTQSPAIMSASPGEKVTMTCRASSSVSYMNWYQQKSGTSPKRWIYDTSKVASGVPY |
| | VL | RFSGSGSGTSYSLTISSMEAEDAATYYCQQWSSNPLTFGAGTKLELK |
| 46 | BCMA-NKG2A | EIVLTQSPGTLSLSPGERATLSCRASQSVSSSYLAWYQQKPGQAPRLLIYGASSRATGIPD |
| | tandem | RFSGSGSGTDFTLTISRLEPEDFAVYYCQQYFNPPEYTFGQGTKVEIKRGGGGSQVQLVQ |
| | fragment 1 | SGAEVKKPGASVKVSCKASGYTFTSYWMNWVRQAPGQGLEWMGRIDPYDSETHYAQ |
| | | KLQGRVTMTTDTSTSTAYMELRSLRSDDTAVYYCARGGYDFDVGTLYWFFDVWGQGT |
| | | TVTVSSGGGGSGGGGSGGGGSDIQMTQSPSSLSASVGDRVTITCRASENIYSYLAWYQQ |
| | | KPGKAPKLLIYNAKTLAEGVPSRFSGSGSGTDFTLTISSLQPEDFATYYCQHHYGTPRTFG |
| | | GGTKVEIKGGGGSEVQLLESGGGLVQPGGSLRLSCAASGFTFGGNAMSWVRQAPGKGL |
| | | EWVSAISGNGGSTFYADSVKGRFTISRDNSKNTLYLQMNSLRAEDTAVYYCAKVRPFWG |
| | | TFDYWGQGTLVTVSS |
| 47 | BCMA-NKG2A | EVQLLESGGGLVQPGGSLRLSCAASGFTFGGNAMSWVRQAPGKGLEWVSAISGNGGST |
| | tandem | FYADSVKGRFTISRDNSKNTLYLQMNSLRAEDTAVYYCAKVRPFWGTFDYWGQGTLVT |
| | fragment 2 | VSSGGGGSGGGGSGGGGSEIVLTQSPGTLSLSPGERATLSCRASQSVSSSYLAWYQQKPG |
| | | QAPRLLIYGASSRATGIPDRFSGSGSGTDFTLTISRLEPEDFAVYYCQQYFNPPEYTFGQGT |
| | | KVEIKRGGGGSGGGGSGGGGSDIQMTQSPSSLSASVGDRVTITCRASENIYSYLAWYQQ |
| | | KPGKAPKLLIYNAKTLAEGVPSRFSGSGSGTDFTLTISSLQPEDFATYYCQHHYGTPRTFG |
| | | GGTKVEIKGGGGGGGGSGGGGSQVQLVQSGAEVKKPGASVKVSCKASGYTFTSYWM |
| | | NWVRQAPGQGLEWMGRIDPYDSETHYAQKLQGRVTMTTDTSTSTAYMELRSLRSDDTA |
| | | VYYCARGGYDFDVGTLYWFFDVWGQGTTVTVSS |
| 48 | BCMA-NKG2A | DIQMTQSPSSLSASVGDRVTITCRASENIYSYLAWYQQKPGKAPKLLIYNAKTLAEGVPS |
| | tandem | RFSGSGSGTDFTLTISSLQPEDFATYYCQHHYGTPRTFGGGTKVEIKGGGGSGGGGSGGG |
| | fragment 3 | GSQVQLVQSGAEVKKPGASVKVSCKASGYTFTSYWMNWVRQAPGQGLEWMGRIDPY |
| | | DSETHYAQKLQGRVTMTTDTSTSTAYMELRSLRSDDTAVYYCARGGYDFDVGTLYWFF |
| | | DVWGQGTTVTVSSGGGGSGGGGSGGGGSEVQLLESGGGLVQPGGSLRLSCAASGFTFG |
| | | GNAMSWVRQAPGKGLEWVSAISGNGGSTFYADSVKGRFTISRDNSKNTLYLQMNSLRA |
| | | EDTAVYYCAKVRPFWGTFDYWGQGTLVTVSSGGGGSGGGGSGGGGSEIVLTQSPGTLS |
| | | LSPGERATLSCRASQSVSSSYLAWYQQKPGQAPRLLIYGASSRATGIPDRFSGSGSGTDFT |
| | | LTISRLEPEDFAVYYCQQYFNPPEYTFGQGTKVEIKR |
| 49 | BCMA-NKG2A | DIQMTQSPSSLSASVGDRVTITCRASENIYSYLAWYQQKPGKAPKLLIYNAKTLAEGVPS |
| | tandem | RFSGSGSGTDFTLTISSLQPEDFATYYCQHHYGTPRTFGGGTKVEIKGGGGSEVQLLESGG |
| | fragment 4 | GLVQPGGSLRLSCAASGFTFGGNAMSWVRQAPGKGLEWVSAISGNGGSTFYADSVKGR |
| | | FTISRDNSKNTLYLQMNSLRAEDTAVYYCAKVRPFWGTFDYWGQGTLVTVSSGGGGSG |
| | | GGGSGGGGSEIVLTQSPGTLSLSPGERATLSCRASQSVSSSYLAWYQQKPGQAPRLLIYG |
| | | ASSRATGIPDRFSGSGSGTDFTLTISRLEPEDFAVYYCQQYFNPPEYTFGQGTKVEIKRGG |
| | | GGSQVQLVQSGAEVKKPGASVKVSCKASGYTFTSYWMNWVRQAPGQGLEWMGRIDP |
| | | YDSETHYAQKLQGRVTMTTDTSTSTAYMELRSLRSDDTAVYYCARGGYDFDVGTLYWF |
| | | FDVWGQGTTVTVSS |
| 50 | BCMA-NKG2A | DIQMTQSPSSLSASVGDRVTITCRASENIYSYLAWYQQKPGKAPKLLIYNAKTLAEGVPS |
| | tandem | RFSGSGSGTDFTLTISSLQPEDFATYYCQHHYGTPRTFGGGTKVEIKGGGGSEVQLLESGG |
| | fragment 5 | GLVQPGGSLRLSCAASGFTFGGNAMSWVRQAPGKGLEWVSAISGNGGSTFYADSVKGR |
| | | FTISRDNSKNTLYLQMNSLRAEDTAVYYCAKVRPFWGTFDYWGQGTLVTVSSGSTSGSG |
| | | KPGSGEGSTKGEIVLTQSPGTLSLSPGERATLSCRASQSVSSSYLAWYQQKPGQAPRLLIY |
| | | GASSRATGIPDRFSGSGSGTDFTLTISRLEPEDFAVYYCQQYFNPPEYTFGQGTKVEIKRG |
| | | GGGSQVQLVQSGAEVKKPGASVKVSCKASGYTFTSYWMNWVRQAPGQGLEWMGRID |
| | | PYDSETHYAQKLQGRVTMTTDTSTSTAYMELRSLRSDDTAVYYCARGGYDFDVGTLYW |
| | | FFDVWGQGTTVTVSS |
| 51 | 28Z amino acid | TTTPAPRPPTPAPTIASQPLSLRPEACRPAAGGAVHTRGLDFACDFWVLVVVGGVLACYS |
| | sequence | LLVTVAFIIFWVRSKRSRLLHSDYMNMTPRRPGPTRKHYQPYAPPRDFAAYRSRVKFSRS |
| | | ADAPAYQQGQNQLYNELNLGRREEYDVLDKRRGRDPEMGGKPQRRKNPQEGLYNELQ |
| | | KDKMAEAYSEIGMKGERRRGKGHDGLYQGLSTATKDTYDALHMQALPPR |
| 52 | BBZ amino acid | TTTPAPRPPTPAPTIASQPLSLRPEACRPAAGGAVHTRGLDFACDIYIWAPLAGTCGVLLLS |
| | sequence | LVITLYCKRGRKKLLYIFKQPFMRPVQTTQEEDGCSCRFPEEEEGGCELRVKFSRSADAPA |
| | | YQQGQNQLYNELNLGRREEYDVLDKRRGRDPEMGGKPQRRKNPQEGLYNELQKDKM |
| | | AEAYSEIGMKGERRRGKGHDGLYQGLSTATKDTYDALHMQALPPR |
| 53 | 28BBZ amino | TTTPAPRPPTPAPTIASQPLSLRPEACRPAAGGAVHTRGLDFACDFWVLVVVGGVLACYS |
| | acid sequence | LLVTVAFIIFWVRSKRSRLLHSDYMNMTPRRPGPTRKHYQPYAPPRDFAAYRSKRGRKK |
| | | LLYIFKQPFMRPVQTTQEEDGCSCRFPEEEEGGCELRVKFSRSADAPAYQQGQNQLYNEL |
| | | NLGRREEYDVLDKRRGRDPEMGGKPQRRKNPQEGLYNELQKDKMAEAYSEIGMKGER |
| | | RRGKGHDGLYQGLSTATKDTYDALHMQALPPR |
| 54 | BCMA-CAR-1 | EVQLLESGGGLVQPGGSLRLSCAASGFTFGGNAMSWVRQAPGKGLEWVSAISGNGGST |
| | | FYADSVKGRFTISRDNSKNTLYLQMNSLRAEDTAVYYCAKVRPFWGTFDYWGQGTLVT |
| | | VSSGGGGSGGGGSGGGGSEIVLTQSPGTLSLSPGERATLSCRASQSVSSSYLAWYQQKPG |
| | | QAPRLLIYGASSRATGIPDRFSGSGSGTDFTLTISRLEPEDFAVYYCQQYFNPPEYTFGQGT |
| | | KVEIKRTTTPAPRPPTPAPTIASQPLSLRPEACRPAAGGAVHTRGLDFACDIYIWAPLAGTC |
| | | GVLLLSLVITLYCKRGRKKLLYIFKQPFMRPVQTTQEEDGCSCRFPEEEEGGCELRVKFSR |
| | | SADAPAYQQGQNQLYNELNLGRREEYDVLDKRRGRDPEMGGKPQRRKNPQEGLYNEL |
| | | QKDKMAEAYSEIGMKGERRRGKGHDGLYQGLSTATKDTYDALHMQALPPR |
| 55 | BCMA-CAR-2 | evqllesggglvqpggslrlscaasgftfggnamswvrqapgkglewvsaisgnggstfyadsvkgrftisrdnskntlyl |
| | | qmnslraedtavyycakvrpfwgtfdywgqgtlvtvssggggsggggsggggseivltqspgtlslspgeratlscrasqs |
| | | vsssylawyqqkpgqaprlliygassratgipdrfsgsgsgtdftltisrlepedfavyycqqyfnppeytfgqgtkveik |
| | | rTTTPAPRPPTPAPTIASQPLSLRPEACRPAAGGAVHTRGLDFACDFWVLVVVGGVLACYSLLVTVAFIIFWVRSKRSR |
| | | LLHSDYMNMTPRRPGPTRKHYQPYAPPRDFAAYRSRVKFSRSADAPAYQQGQNQLYNE |
| | | LNLGRREEYDVLDKRRGRDPEMGGKPQRRKNPQEGLYNELQKDKMAEAYSEIGMKGE |
| | | RRRGKGHDGLYQGLSTATKDTYDALHMQALPPR |
| 56 | BCMA-CAR-3 | evqllesggglvqpggslrlscaasgftfggnamswvrqapgkglewvsaisgnggstfyadsvkgrftisrdnskntlyl |
| | | qmnslraedtavyycakvrpfwgtfdywgqgtlvtvssggggsggggsggggseivltqspgtlslspgeratlscrasqs |
| | | vsssylawyqqkpgqaprlliygassratgipdrfsgsgsgtdftltisrlepedfavyycqqyfnppeytfgqgtkveik |
| | | rTTTPAPRPPTPAPTIASQPLSLRPEACRPAAGGAVHTRGLDFACDFWVLVVVGGVLACYSLLVTVAFIIFWVRSKRSR |
| | | LLHSDYMNMTPRRPGPTRKHYQPYAPPRDFAAYRSKRGRKKLLYIFKQPFMRPVQTTQE |
| | | EDGCSCRFPEEEEGGCELRVKFSRSADAPAYQQGQNQLYNELNLGRREEYDVLDKRRGR |
| | | DPEMGGKPQRRKNPQEGLYNELQKDKMAEAYSEIGMKGERRRGKGHDGLYQGLSTAT |
| | | KDTYDALHMQALPPR |
| 57 | NKG2A-CAR | QVQLVQSGAEVKKPGASVKVSCKASGYTFTSYWMNWVRQAPGQGLEWMGRIDPYDS |
| | | ETHYAQKLQGRVTMTTDTSTSTAYMELRSLRSDDTAVYYCARGGYDFDVGTLYWFFDV |
| | | WGQGTTVTVSSGGGGSGGGGSGGGGSDIQMTQSPSSLSASVGDRVTITCRASENIYSYL |
| | | AWYQQKPGKAPKLLIYNAKTLAEGVPSRFSGSGSGTDFTLTISSLQPEDFATYYCQHHYG |
| | | TPRTFGGGTKVEIKTTTPAPRPPTPAPTIASQPLSLRPEACRPAAGGAVHTRGLDFACDFW |
| | | VLVVVGGVLACYSLLVTVAFIIFWVRSKRSRLLHSDYMNMTPRRPGPTRKHYQPYAPPR |
| | | DFAAYRSRVKFSRSADAPAYQQGQNQLYNELNLGRREEYDVLDKRRGRDPEMGGKPQR |
| | | RKNPQEGLYNELQKDKMAEAYSEIGMKGERRRGKGHDGLYQGLSTATKDTYDALHMQ |
| | | ALPPR |
| 58 | BCMA-NKG2A | EIVLTQSPGTLSLSPGERATLSCRASQSVSSSYLAWYQQKPGQAPRLLIYGASSRATGIPD |
| | tandem | RFSGSGSGTDFTLTISRLEPEDFAVYYCQQYFNPPEYTFGQGTKVEIKRGGGGSQVQLVQ |
| | fragment-CAR | SGAEVKKPGASVKVSCKASGYTFTSYWMNWVRQAPGQGLEWMGRIDPYDSETHYAQ |
| | | KLQGRVTMTTDTSTSTAYMELRSLRSDDTAVYYCARGGYDFDVGTLYWFFDVWGQGT |
| | | TVTVSSGGGGSGGGGSGGGGSDIQMTQSPSSLSASVGDRVTITCRASENIYSYLAWYQQ |
| | | KPGKAPKLLIYNAKTLAEGVPSRFSGSGSGTDFTLTISSLQPEDFATYYCQHHYGTPRTFG |
| | | GGTKVEIKGGGGSEVQLLESGGGLVQPGGSLRLSCAASGFTFGGNAMSWVRQAPGKGL |
| | | EWVSAISGNGGSTFYADSVKGRFTISRDNSKNTLYLQMNSLRAEDTAVYYCAKVRPFWG |
| | | TFDYWGQGTLVTVSSTTTPAPRPPTPAPTIASQPLSLRPEACRPAAGGAVHTRGLDFACDI |
| | | YIWAPLAGTCGVLLLSLVITLYCKRGRKKLLYIFKQPFMRPVQTTQEEDGCSCRFPEEEEG |
| | | GCELRVKFSRSADAPAYQQGQNQLYNELNLGRREEYDVLDKRRGRDPEMGGKPQRRK |
| | | NPQEGLYNELQKDKMAEAYSEIGMKGERRRGKGHDGLYQGLSTATKDTYDALHMQAL |
| | | PPR |
| 59 | NKG2A-CD3 | DIQMTQSPSSLSASVGDRVTITCRASENIYSYLAWYQQKPGKAPKLLIYNAKTLAEGVPS |
| | amino acid | RFSGSGSGTDFTLTISSLQPEDFATYYCQHHYGTPRTFGGGTKVEIKGGGGSGGGGSGGG |
| | sequence | GSQVQLVQSGAEVKKPGASVKVSCKASGYTFTSYWMNWVRQAPGQGLEWMGRIDPY |
| | | DSETHYAQKLQGRVTMTTDTSTSTAYMELRSLRSDDTAVYYCARGGYDFDVGTLYWFF |
| | | DVWGQGTTVTVSSGGGSDIKLQQSGAELARPGASVKMSCKTSGYTFTRYTMHWVKQR |
| | | PGQGLEWIGYINPSRGYTNYNQKFKDKATLTTDKSSSTAYMQLSSLTSEDSAVYYCARY |
| | | YDDHYCLDYWGQGTTLTVSSVEGGSGGSGGSGGSGGVDDIQLTQSPAIMSASPGEKVT |
| | | MTCRASSSVSYMNWYQQKSGTSPKRWIYDTSKVASGVPYRFSGSGSGTSYSLTISSMEA |
| | | EDAATYYCQQWSSNPLTFGAGTKLELK |
| 60 | A1-BITE amino | DIQMTQSPSTLSASVGDRVTITCRASQSISSWLAWYQQKPGKAPKLLIYDASSLESGVPSR |
| | acid sequence | FSGSGSGTEFTLTISSLQPDDFATYYCQQYDSYVSTFGQGTKVEIKRGGGGSGGGGSGGG |
| | | GSEVQLLESGGGLVQPGGSLRLSCAASGFTFNRFYMSWVRQAPGKGLEWVSAITGWGG |
| | | STYYADSVKGRFTISRDNSKNTLYLQMNSLRAEDTAVYYCAKGYDGFDYWGQGTLVTV |
| | | SSGGGSDIKLQQSGAELARPGASVKMSCKTSGYTFTRYTMHWVKQRPGQGLEWIGYIN |
| | | PSRGYTNYNQKFKDKATLTTDKSSSTAYMQLSSLTSEDSAVYYCARYYDDHYCLDYWG |
| | | QGTTLTVSSVEGGSGGSGGSGGSGGVDDIQLTQSPAIMSASPGEKVTMTCRASSSVSYM |
| | | NWYQQKSGTSPKRWIYDTSKVASGVPYRFSGSGSGTSYSLTISSMEAEDAATYYCQQWS |
| | | SNPLTFGAGTKLELK |
| 61 | A2-BITE amino | DIQMTQSPSTLSASVGDRVTITCRASQSISSWLAWYQQKPGKAPKLLIYDASSLESGVPSR |
| | acid sequence | FSGSGSGTEFTLTISSLQPDDFATYYCQQYDSYVSTFGQGTKVEIKRGGGGSGGGGSGGG |
| | | GSEVQLLESGGGLVQPGGSLRLSCAASGFTFGRVHMSWVRQAPGKGLEWVSAISAGGG |
| | | STYYADSVKGRFTISRDNSKNTLYLQMNSLRAEDTAVYYCAKGYDGFDYWGQGTLVTV |
| | | SSGGGSDIKLQQSGAELARPGASVKMSCKTSGYTFTRYTMHWVKQRPGQGLEWIGYIN |
| | | PSRGYTNYNQKFKDKATLTTDKSSSTAYMQLSSLTSEDSAVYYCARYYDDHYCLDYWG |
| | | QGTTLTVSSVEGGSGGSGGSGGSGGVDDIQLTQSPAIMSASPGEKVTMTCRASSSVSYM |
| | | NWYQQKSGTSPKRWIYDTSKVASGVPYRFSGSGSGTSYSLTISSMEAEDAATYYCQQWS |
| | | SNPLTFGAGTKLELK |
| 62 | A3-BITE amino | DIQMTQSPSTLSASVGDRVTITCRASQSISSWLAWYQQKPGKAPKLLIYDASSLESGVPSR |
| | acid sequence | FSGSGSGTEFTLTISSLQPDDFATYYCQQYDSYVSTFGQGTKVEIKRGGGGSGGGGSGGG |
| | | GSEVQLLESGGGLVQPGGSLRLSCAASGFTFRNFHVSWVRQAPGKGLEWVSAINGPVGS |
| | | TYYADSVKGRFTISRDNSKNTLYLQMNSLRAEDTAVYYCAKGYDGFDYWGQGTLVTVS |
| | | SGGGSDIKLQQSGAELARPGASVKMSCKTSGYTFTRYTMHWVKQRPGQGLEWIGYINP |
| | | SRGYTNYNQKFKDKATLTTDKSSSTAYMQLSSLTSEDSAVYYCARYYDDHYCLDYWGQ |
| | | GTTLTVSSVEGGSGGSGGSGGSGGVDDIQLTQSPAIMSASPGEKVTMTCRASSSVSYMN |
| | | WYQQKSGTSPKRWIYDTSKVASGVPYRFSGSGSGTSYSLTISSMEAEDAATYYCQQWSS |
| | | NPLTFGAGTKLELK |
| 63 | NKP46-CD3 | DIQMTQSPSSLSASLGERVSLTCRASQDIGSSLNWLQQEPDGTIKRLIYATSRLDSGVPKRF |
| | amino acid | SGSRSGSDYSLTISSLESEDFVDYYCLQYASSPWTFGGGTKLEIKRGGGGSGGGGSGGGG |
| | sequence | SQVQLQQPGSVLVRPGASVKLSCKASGYTFTSSWMHWAKQRPGQGLEWIGHIHPNSGIS |
| | | NYNEKFKGKATLTVDTSSSTAYVDLSSLTSEDSAVYYCSRGGRFDDWGAGTTVTVSSGG |
| | | GSDIKLQQSGAELARPGASVKMSCKTSGYTFTRYTMHWVKQRPGQGLEWIGYINPSRG |
| | | YTNYNQKFKDKATLTTDKSSSTAYMQLSSLTSEDSAVYYCARYYDDHYCLDYWGQGTT |
| | | LTVSSVEGGSGGSGGSGGSGGVDDIQLTQSPAIMSASPGEKVTMTCRASSSVSYMNWYQ |
| | | QKSGTSPKRWIYDTSKVASGVPYRFSGSGSGTSYSLTISSMEAEDAATYYCQQWSSNPLT |
| | | FGAGTKLELK |
| 64 | g-TRAC-2 | TCTCTCAGCTGGTACACGGC |
| 65 | g-TRAC-3 | GAGAATCAAAATCGGTGAAT |
| 66 | g-B2M-2 | GGCCACGGAGCGAGACATCT |
| 67 | g-B2M-3 | CGCGAGCACAGCTAAGGCCA |

1. A bispecific molecule, wherein the molecule comprises:
a first binding domain that binds to a NK cell receptor on the surface of a target cell; and
a second binding domain that binds to a CD3 on the surface of a T cell,
preferably, wherein the NK cell receptor comprises a NK inhibitory receptor and/or a NK activating receptor,
more preferably, the NK cell receptor comprises a NKG2A and/or a NKP46.

2. (canceled)

3. (canceled)

4. The molecule according to claim 1, wherein
the first binding domain binds to human or macaque NKG2A and/or NKP46; and/or
the second binding domain binds to human CD3ε, Callithrix jacchus CD3ε, Saguinus oedipus CD3ε, or Saimiri sciureus CD3ε,
preferably, wherein the molecule is any one selected from the group consisting of: scFv, (scFv) 2, scFv-single domain antibody, bifunctional antibody, and an oligomer thereof.

5. (canceled)

6. The molecule according to claim 1, wherein
the first binding domain comprises a sequence represented by SEQ ID NO:34 and/or SEQ ID NO: 35, or an amino acid sequence that is at least 90%, 91%, 92%, 93%, 94%, 95%, 96%, 97%, 98%, 99% or more identical to the amino acid sequence of SEQ ID NO:34 or 35, or a sequence represented by SEQ ID NO:36 and/or SEQ ID NO:37, or an amino acid sequence that is at least 90%, 91%, 92%, 93%, 94%, 95%, 96%, 97%, 98%, 99% or more identical to the amino acid sequence of SEQ ID NO:36 or 37, or a sequence represented by SEQ ID NO:38 and/or SEQ ID NO:39, or an amino acid sequence that is at least 90%, 91%, 92%, 93%, 94%, 95%, 96%, 97%, 98%, 99% or more identical to the amino acid sequence of SEQ ID NO:38 or 39, or an amino acid sequence represented by SEQ ID NO:40 and/or SEQ ID NO:41, or an amino acid sequence that is at least 90%, 91%, 92%, 93%, 94%, 95%, 96%, 97%, 98%, 99% or more identical to the amino acid sequence of SEQ ID NO:40 or 41, or a sequence represented by SEQ ID NO:42 and/or SEQ ID NO:43, or an amino acid sequence that is at least 90%, 91%, 92%, 93%, 94%, 95%, 96%, 97%, 98%, 99% or more identical to the amino acid sequence of SEQ ID NO:42 or 43;
the second binding domain comprises a sequence represented by SEQ ID NO:44 and SEQ ID NO: 45, or an amino acid sequence that is at least 90%, 91%, 92%, 93%, 94%, 95%, 96%, 97%, 98%, 99% or more identical to the amino acid sequence of SEQ ID NO:44 or 45.

7. The molecule according to claim 1, wherein
the molecule comprises an amino acid sequence represented by SEQ ID NO: 59, 60, 61, 62 or 63, or an amino acid sequence that is at least 90%, 91%, 92%, 93%, 94%, 95%, 96%, 97%, 98%, 99% or more identical to an amino acid sequence of SEQ ID NO: 59, 60, 61, 62 or 63.

8. (canceled)

9. (canceled)

10. An engineered cell, which secrets the molecule according to claim 1.

11. The engineered cell according to claim 10, wherein the cell further expresses an exogenous receptor that recognizes a tumor antigen and/or a pathogen antigen;
preferably, the tumor antigen is selected from any one of: BCMA, CD19, GPC3, Claudin18.2, EGFR, EGFRVIII, or a combination thereof,
more preferably, wherein the engineered cell is selected from: immune cell, neuron, epithelial cell, endothelial cell or stem cell; preferably, the engineered cell is selected from: T cell, NK cell, cytotoxic T cell, NKT cell, macrophage, CIK cell, and stem cell-derived immune cell, or a combination thereof,
more preferably the endogenous HLA-I, TCR, HLA-II and/or NKG2A genes of the engineered cell are knocked out, preferably by CRISPR/Cas9 technology.

12. (canceled)

13. (canceled)

14. A pharmaceutical composition, comprising: the molecule according to claim 1,
preferably, the composition further comprises T cells expressing a chimeric receptor that recognizes a tumor antigen and/or a pathogen antigen, preferably, the chimeric receptor recognizes any one of BCMA, CD19, GPC3, Claudin18.2, EGFR, EGFRVIII, or a combination thereof.

15-19. (canceled)

20. A method for increasing the durability and/or transplant survival rate of allogeneic immune cells in the presence of host NK cells, which comprises administering to a subject in need thereof the molecule according to claim 1.

21. (canceled)

22. A gRNA construct comprising a first gRNA targeting CIITA, wherein the first gRNA comprises a sequence represented by SEQ ID NO: 1, 2, 3, 4, 5, 6, 7, 8, 9, 10, 11, 12 or 13; or a gRNA construct comprising a second gRNA targeting NKG2A, wherein the second gRNA comprises a sequence represented by SEQ ID NO: 14, 15 or 21;
preferably, wherein the first gRNA comprises a sequence of 16, 17, 18 or 19 continuous nucleotides in the sequence represented by SEQ ID NO: 4, 12 or 13; and/or the second gRNA comprises a sequence of 16, 17, 18 or 19 continuous nucleotides in the sequence represented by SEQ ID NO: 14, 15, 21 or 23.

23. (canceled)

24. (canceled)

25. The construct according to claim 22, further comprising a third gRNA targeting TRAC gene and/or a fourth gRNA targeting B2M gene;
preferably, wherein the third gRNA comprises a sequence represented by SEQ ID NOs: 24, 64 and/or 65; and/or the fourth gRNA comprises a sequence represented by SEQ ID NOs: 25, 66 and/or 67,
more preferably, wherein the first, second, third and fourth gRNAs comprise the sequences represented by SEQ ID NOs: 4, 14, 24 and 25, respectively; or the first, second, third and fourth gRNAs comprise the sequences represented by SEQ ID NOs: 4, 15, 24 and 25, respectively; or the first, second, third and fourth gRNAs comprise the sequences represented by SEQ ID NOs: 4, 23, 24 and 25, respectively; or the first, second, third and fourth gRNAs comprise the sequences represented by SEQ ID NOs: 12, 23, 24 and 25, respectively; or the first, second, third and fourth gRNAs comprise the sequences represented by SEQ ID NOs: 12, 15, 24 and 25, respectively; or the first, second, third and fourth gRNAs comprise the sequences represented by SEQ ID NOs: 13, 23, 24 and 25, respectively; or the first, second, third, and fourth gRNAs comprise the sequences represented by SEQ ID NOs: 13, 15, 24 and 25, respectively; the first, second, third and fourth gRNAs comprise the sequences represented by SEQ ID NOs: NO: 4, 14, 24 and 66; or the first, second, third and fourth gRNAs comprise the sequences represented by SEQ ID NOs: 4, 15, 24 and 66, respectively; or the first, second, third, and fourth gRNAs comprise the sequences represented by SEQ ID NOs: 4, 23, 24 and 66, respectively; or the first, second, third and fourth gRNAs comprise the sequences represented by SEQ ID NOs: 12, 23, 24 and 66, respectively; or the first, second, third and fourth gRNAs comprise the sequences represented by SEQ ID NOs: 12, 15, 24 and 66, respectively; or the first, second, third and fourth gRNAs comprise the sequences represented by SEQ ID NOs: 13, 23, 24 and 66, respectively; or the first, second, third and fourth gRNAs comprise sequences represented by SEQ ID NOs: 13, 15, 24 and 66, respectively.

26. (canceled)

27. (canceled)

28. The construct according to claim 22, wherein it comprises a first, a second, a third or a fourth gRNA connected to crRNA/tracrRNA, respectively,
preferably, wherein the crRNA/tracrRNA comprises a sequence represented by SEQ ID NO: 26.

29. (canceled)

30. A method for gene editing of CIITA or NKG2A in cells by the CRISPR/Cas system, which comprises conducting gene editing of the cells by using the construct according to claim 22.

31. (canceled)

32. The method according to claim 30, wherein the Cas protein in the CRISPR/Cas system is selected from the group consisting of: Cas9 protein, Cas12a protein, Cas12b protein, Cas12c protein, Cas12d protein, Cas12e protein, Cas12f protein, Cas12g protein, Cas12h protein, Cas12i protein, Cas14 protein, Cas13a protein, Cas1 protein, Cas1B protein, Cas2 protein, Cas3 protein, Cas4 protein, Cas5 protein, Cas6 protein, Cas7 protein, Cas8 protein, Cas10 protein, Csy1 protein, Csy2 protein, Csy3 protein, Cse1 protein, Cse2 protein, Csc1 protein, Csc2 protein, Csa5 protein, Csn2 protein, Csm2 protein, Csm3 protein, Csm4 protein, Csm5 protein, Csm6 protein, Cmr1 protein, Cmr3 protein, Cmr4 protein, Cmr5 protein, Cmr6 protein, Csb1 protein, Csb2 protein, Csb3 protein, Csx17 protein, Csx14 protein, Csx10 protein, Csx16 protein, CsaX protein, Csx3 protein, Csx1 protein, Csx15 protein, Csf1 protein, Csf2 protein, Csf3 protein, Csf4 protein, and homologs or modified forms thereof,
preferably, wherein a complex of a nucleic acid and a protein comprising a Cas protein mixed with the gRNA is simultaneously introduced into the cells for gene editing,
more preferably, wherein a molar ratio of the Cas9 protein to the gRNA is 1:1 to 1:10, preferably 1:3 to 1:5, and further preferably 1:4.

33. (canceled)

34. (canceled)

35. The method according to claim 30, wherein the cells are selected from: T cells, NK cells, cytotoxic T cells, NKT cells, macrophages, CIK cells, stem cells, and stem cell-derived immune cells, or a combination thereof,
preferably, wherein the cells are selected from: autologous T cells or allogeneic T cells, stem cell-derived T cells, primary T cells or autologous T cells derived from human.

36-38. (canceled)

39. A cell, wherein the cell comprises:
a) low or no expression of endogenous CIITA molecules and/or NKG2A molecules;
b) low or no expression of endogenous TCR/B2M/NKG2A molecules;
c) low or no expression of endogenous TCR/B2M/CIITA molecules; and/or
d) low or no expression of endogenous TCR/B2M/CIITA/NKG2A molecules,
preferably, wherein the endogenous TCR, B2M, CIITA and/or NKG2A molecules are knocked out by CRISPR/Cas9 technology.

40. (canceled)

41. The cell according to claim 39, wherein the cell is engineered by using a gRNA construct comprising a first gRNA targeting CIITA, wherein the first gRNA comprises a sequence represented by SEQ ID NO: 1, 2, 3, 4, 5, 6, 7, 8, 9, 10, 11, 12 or 13; or a gRNA construct comprising a second gRNA targeting NKG2A, wherein the second gRNA comprises a sequence represented by SEQ ID NO: 14, 15 or 21;
preferably, wherein the first gRNA comprises a sequence of 16, 17, 18 or 19 continuous nucleotides in the sequence represented by SEQ ID NO: 4, 12 or 13; and/or the second gRNA comprises a sequence of 16, 17, 18 or 19 continuous nucleotides in the sequence represented by SEQ ID NO: 14, 15, 21 or 23,
preferably, wherein the gRNA used in the CRISPR/Cas9 technology comprises: the sequences represented by SEQ ID NOs: 4, 14, 24 and 25; or the sequences represented by SEQ ID NOs: 4, 15, 24 and 25; or the sequences represented by SEQ ID NOs: 4, 23, 24 and 25; or the sequences represented by SEQ ID NOs: 12, 14, 24 and 25; or the sequences represented by SEQ ID NOs: 12, 15, 24 and 25; or the sequences represented by SEQ ID NOs: 12, 23, 24 and 25; or the sequences represented by SEQ ID NOs: 13, 14, 24 and 25; or the sequences represented by SEQ ID NOs: 13, 15, 24 and 25; or the sequences represented by SEQ ID NOs: 13, 23, 24 and 25; or the sequences represented by SEQ ID NOs: 4 and/or 14; or the sequences represented by SEQ ID NOs: 4 and/or 15; or the sequences represented by SEQ ID NOs: 4 and/or 23; or the sequences represented by SEQ ID NOs: NO: 4, 24 and 25; or the sequences represented by SEQ ID NO: 12 and/or 14; or the sequences represented by SEQ ID NO: 12 and/or 15; or the sequences represented by SEQ ID NO: 12 and/or 23; or the sequences represented by SEQ ID NOs: 12, 24 and 25; or the sequences represented by SEQ ID NO: 13 and/or 14; or the sequences represented by SEQ ID NO: 13 and/or 15; or the sequences represented by SEQ ID NO: 13 and/or 23; or the sequences represented by SEQ ID NOs: 13, 24 and 25; or the sequences represented by SEQ ID NOs: 14, 24 and 25; or the sequences represented by SEQ ID NOs: 15, 24 and 25; or the sequence represented by SEQ ID NOs: 23, 24 and 25; or the sequences represented by SEQ ID NOs: 4, 14, 24 and 66; or the sequences represented by SEQ ID NOs: 4, 15, 24 and 66; or the sequences represented by SEQ ID NOs: NO: 4, 23, 24 and 66; or the sequences represented by SEQ ID NOs: 12, 14, 24 and 66; or the sequences represented by SEQ ID NOs: 12, 15, 24 and 66; or the sequences represented by SEQ ID NOs: 12, 23, 24 and 66; or the sequences represented by SEQ ID NOs: 13, 14, 24 and 66; or the sequences represented by SEQ ID NOs: 13, 15, 24 and 66; or the sequences represented by SEQ ID NOs: 13, 23, 24 and 66; or the sequences represented by SEQ ID NOs: 4, 24 and 66; or the sequences represented by SEQ ID NOs: 12, 24 and 66; or the sequences represented by SEQ ID NOs: 13, 24 and 66; or the sequences represented by SEQ ID NOs: 14, 24 and 66; or the sequences represented by SEQ ID NOs: 15, 24 and 66; or the sequences represented by SEQ ID NOs: 23, 24 and 66.

42. (canceled)

43. The cell according to claim 39, wherein the cell further expresses an exogenous receptor that recognizes an NK cell receptor, a tumor antigen and/or a pathogen antigen, preferably expresses an exogenous receptor that recognizes a NKG2A,
preferably, wherein the exogenous receptor comprises a chimeric antigen receptor (CAR) or a recombinant TCR receptor;
more preferably, wherein the CAR comprises:
a) an antibody that recognizes a NKG2A polypeptide, a tumor and/or a pathogen antigen, a transmembrane region of CD28 or CD8, a co-stimulatory signaling domain of CD28, and CD3δ; and/or
b) an antibody that recognizes a NKG2A polypeptide, a tumor and/or a pathogen antigen, a transmembrane region of CD28 or CD8, a co-stimulatory signaling domains of CD137, and CD3δ; and/or
c) an antibody that recognizes a NKG2A polypeptide, a tumor and/or a pathogen antigen, a transmembrane region of CD28 or CD8, a costimulatory signaling domain of CD28, a costimulatory signaling domain of CD137, and CD3δ; or
d) an antibody that recognizes a NKG2A polypeptide, a tumor and/or a pathogen antigen, a transmembrane region of CD28 or CD8, and CD3δ,
preferably, wherein the cell is selected from the group consisting of: T cells, NK cells, cytotoxic T cells, NKT cells, macrophages, CIK cells, stem cells, and stem cell-derived immune cells, or a combination thereof,
more preferably, wherein the cell is selected from: autologous T cells or allogeneic T cells, stem cell-derived T cells, primary T cells, or autologous T cells derived from human.

44-47. (canceled)

48. The cell according to claim 43, wherein the tumor antigen is selected from: CD19, GPC3, Claudin 18.2, WT1, HER2, EGFR, BCMA, or a combination thereof,
preferably, the antibody recognizing the NKG2A polypeptide comprises: the heavy chain variable region and the light chain variable region respectively represented by SEQ ID NO: 34 and SEQ ID NO: 35, the heavy chain variable region and the light chain variable region respectively represented by SEQ ID NO: 36 and SEQ ID NO: 37, the heavy chain variable region and the light chain variable region respectively represented by SEQ ID NO: 38 and SEQ ID NO: 39, or the heavy chain variable region and the light chain variable region respectively represented by SEQ ID NO: 40 and SEQ ID NO: 41; or a tandem antibody sequence represented by SEQ ID NO: 46, 47, 48, 49 or 50,
more preferably, the antibody recognizing a tumor antigen comprises: a heavy chain variable region and a light chain variable region respectively represented by SEQ ID NO: 27 and SEQ ID NO: 28; or a scFv represented by SEQ ID NO: 29, 30, 31, 32 or 33; or a tandem antibody sequence represented by SEQ ID NO: 46, 47, 48, 49 or 50.

49-55. (canceled)

56. A pharmaceutical composition, comprising: the engineered cell according to claim 10; preferably, the composition further comprises T cells expressing a chimeric receptor that recognizes a tumor antigen and/or a pathogen antigen, preferably, the chimeric receptor recognizes any one of BCMA, CD19, GPC3, Claudin18.2, EGFR, EGFRvIII, or a combination thereof.

57. A method for increasing the durability and/or transplant survival rate of allogeneic immune cells in the presence of host NK cells, which comprises administering to a subject in need thereof the engineered cell according to claim 10.


SEQUENCE LISTING (67 sequences)

SEQ ID NO: 1 - RNA, 20 nt, synthetic construct
  source 1..20: mol_type = other RNA
gctgaactggtcgcagttga

SEQ ID NO: 2 - RNA, 20 nt, synthetic construct
  source 1..20: mol_type = other RNA
gatattggcataagcctccc

SEQ ID NO: 3 - RNA, 20 nt, synthetic construct
  source 1..20: mol_type = other RNA
tcaactgcgaccagttcagc

SEQ ID NO: 4 - RNA, 20 nt, synthetic construct
  source 1..20: mol_type = other RNA
gagaagacaaagtcgtactg

SEQ ID NO: 5 - RNA, 20 nt, synthetic construct
  source 1..20: mol_type = other RNA
gccattgcttgaaccgtccg

SEQ ID NO: 6 - RNA, 20 nt, synthetic construct
  source 1..20: mol_type = other RNA
gccacagccctactttgtgc

SEQ ID NO: 7 - RNA, 20 nt, synthetic construct
  source 1..20: mol_type = other RNA
ggtccatctggtcatagaag

SEQ ID NO: 8 - RNA, 20 nt, synthetic construct
  source 1..20: mol_type = other RNA
gagattcaggcagctcaacg

SEQ ID NO: 9 - RNA, 20 nt, synthetic construct
  source 1..20: mol_type = other RNA
ggacagctcaaatagggcgt

SEQ ID NO: 10 - RNA, 19 nt, synthetic construct
  source 1..19: mol_type = other RNA
catcaaagtaccctacagg

SEQ ID NO: 11 - RNA, 19 nt, synthetic construct
  source 1..19: mol_type = other RNA
agagtcccgtgagcgtgga

SEQ ID NO: 12 - RNA, 19 nt, synthetic construct
  source 1..19: mol_type = other RNA
gtctaggatgagcagaacg

SEQ ID NO: 13 - RNA, 19 nt, synthetic construct
  source 1..19: mol_type = other RNA
cacgagtgattgctgtgct

SEQ ID NO: 14 - RNA, 20 nt, synthetic construct
  source 1..20: mol_type = other RNA
agataagacagataattccc

SEQ ID NO: 15 - RNA, 20 nt, synthetic construct
  source 1..20: mol_type = other RNA
tgaacaggaaataacctatg

SEQ ID NO: 16 - RNA, 20 nt, synthetic construct
  source 1..20: mol_type = other RNA
aaaccattcattgtcaccca

SEQ ID NO: 17 - RNA, 20 nt, synthetic construct
  source 1..20: mol_type = other RNA
atattattgaagatccacac

SEQ ID NO: 18 - RNA, 20 nt, synthetic construct
  source 1..20: mol_type = other RNA
actgcagagatggataacca

SEQ ID NO: 19 - RNA, 20 nt, synthetic construct
  source 1..20: mol_type = other RNA
gacaaaacctatcactgcaa

SEQ ID NO: 20 - RNA, 20 nt, synthetic construct
  source 1..20: mol_type = other RNA
ctgaatacaagaactcagaa

SEQ ID NO: 21 - RNA, 20 nt, synthetic construct
  source 1..20: mol_type = other RNA
atgggtgacaatgaatggtt

SEQ ID NO: 22 - RNA, 20 nt, synthetic construct
  source 1..20: mol_type = other RNA
tccaacagttgttactacat

SEQ ID NO: 23 - RNA, 20 nt, synthetic construct
  source 1..20: mol_type = other RNA
ggtctgagtagattactcct

SEQ ID NO: 24 - RNA, 20 nt, synthetic construct
  source 1..20: mol_type = other RNA
agagtctctcagctggtaca

SEQ ID NO: 25 - RNA, 20 nt, synthetic construct
  source 1..20: mol_type = other RNA
gagtagcgcgagcacagcta

SEQ ID NO: 26 - RNA, 80 nt, synthetic construct
  source 1..80: mol_type = other RNA
gttttagagctagaaatagcaagttaaaataaggctagtccgttatcaacttgaaaaagtggcaccgagtcggtgctttt

SEQ ID NO: 27 - protein, 119 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSS

SEQ ID NO: 28 - protein, 110 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 29 - protein, 244 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 30 - protein, 242 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCARYPYLAFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGYPPSYTFGQGTKVEIKR

SEQ ID NO: 31 - protein, 243 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKLSGDAAMDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYGYPPRYTFGQGTKVEIKR

SEQ ID NO: 32 - protein, 244 aa, synthetic construct
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 33 - protein, 244 aa, synthetic construct
EVQLLESGGGLVQPGGSLRLSCAASGFTFRSYAMSWVRQAPGKGLEWVSAISGGGGNTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 34 - protein, 125 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 35 - protein, 107 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIK

SEQ ID NO: 36 - protein, 116 aa, synthetic construct
EVQLLESGGGLVQPGGSLRLSCAASGFTFNRFYMSWVRQAPGKGLEWVSAITGWGGSTYYADSVKGRFTISRDNSKNTLYLQMNSLRAEDTAVYYCAKGYDGFDYWGQGTLVTVSS

SEQ ID NO: 37 - protein, 108 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDSYVSTFGQGTKVEIKR

SEQ ID NO: 38 - protein, 116 aa, synthetic construct
EVQLLESGGGLVQPGGSLRLSCAASGFTFGRVHMSWVRQAPGKGLEWVSAISAGGGSTYYADSVKGRFTISRDNSKNTLYLQMNSLRAEDTAVYYCAKGYDGFDYWGQGTLVTVSS

SEQ ID NO: 39 - protein, 108 aa, synthetic construct; identical to SEQ ID NO: 37

SEQ ID NO: 40 - protein, 116 aa, synthetic construct
EVQLLESGGGLVQPGGSLRLSCAASGFTFRNFHVSWVRQAPGKGLEWVSAINGPVGSTYYADSVKGRFTISRDNSKNTLYLQMNSLRAEDTAVYYCAKGYDGFDYWGQGTLVTVSS

SEQ ID NO: 41 - protein, 108 aa, synthetic construct; identical to SEQ ID NO: 37

SEQ ID NO: 42 - protein, 115 aa, synthetic construct
QVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCSRGGRFDDWGAGTTVTVSS

SEQ ID NO: 43 - protein, 108 aa, synthetic construct
DIQMTQSPSSLSASLGERVSLTCRASQDIGSSLNWLQQEPDGTIKRLIYATSRLDSGVPKRFSGSRSGSDYSLTISSLESEDFVDYYCLQYASSPWTFGGGTKLEIKR

SEQ ID NO: 44 - protein, 119 aa, synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSS

SEQ ID NO: 45 - protein, 106 aa, synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 46 - protein, 486 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGGSGGGGSGGGGSDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSS

SEQ ID NO: 47 - protein, 506 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRGGGGSGGGGSGGGGSDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSGGGGSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 48 - protein, 506 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSGGGGSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGGSGGGGSGGGGSEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKR

SEQ ID NO: 49 - protein, 486 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 50 - protein, 489 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGSTSGSGKPGSGEGSTKGEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSS

SEQ ID NO: 51 - protein, 226 aa, synthetic construct
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 52 - protein, 224 aa, synthetic construct
TTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 53 - protein, 268 aa, synthetic construct
TTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 54 - protein, 468 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 55 - protein, 470 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 56 - protein, 512 aa, synthetic construct
EVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSGGGGSGGGGSGGGGSEIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 57 - protein, 473 aa, synthetic construct
QVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGGSGGGGSGGGGSDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 58 - protein, 710 aa, synthetic construct
EIVLTQSPGTLSLSPGERATLSCRASQSVSSSYLAWYQQKPGQAPRLLIYGASSRATGIPDRFSGSGSGTDFTLTISRLEPEDFAVYYCQQYFNPPEYTFGQGTKVEIKRGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGGSGGGGSGGGGSDIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSEVQLLESGGGLVQPGGSLRLSCAASGFTFGGNAMSWVRQAPGKGLEWVSAISGNGGSTFYADSVKGRFTISRDNSKNTLYLQMNSLRAEDTAVYYCAKVRPFWGTFDYWGQGTLVTVSSTTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 59 - protein, 494 aa, synthetic construct
DIQMTQSPSSLSASVGDRVTITCRASENIYSYLAWYQQKPGKAPKLLIYNAKTLAEGVPSRFSGSGSGTDFTLTISSLQPEDFATYYCQHHYGTPRTFGGGTKVEIKGGGGSGGGGSGGGGSQVQLVQSGAEVKKPGASVKVSCKASGYTFTSYWMNWVRQAPGQGLEWMGRIDPYDSETHYAQKLQGRVTMTTDTSTSTAYMELRSLRSDDTAVYYCARGGYDFDVGTLYWFFDVWGQGTTVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 60 - protein, 486 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDSYVSTFGQGTKVEIKRGGGGSGGGGSGGGGSEVQLLESGGGLVQPGGSLRLSCAASGFTFNRFYMSWVRQAPGKGLEWVSAITGWGGSTYYADSVKGRFTISRDNSKNTLYLQMNSLRAEDTAVYYCAKGYDGFDYWGQGTLVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 61 - protein, 486 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDSYVSTFGQGTKVEIKRGGGGSGGGGSGGGGSEVQLLESGGGLVQPGGSLRLSCAASGFTFGRVHMSWVRQAPGKGLEWVSAISAGGGSTYYADSVKGRFTISRDNSKNTLYLQMNSLRAEDTAVYYCAKGYDGFDYWGQGTLVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 62 - protein, 486 aa, synthetic construct
DIQMTQSPSTLSASVGDRVTITCRASQSISSWLAWYQQKPGKAPKLLIYDASSLESGVPSRFSGSGSGTEFTLTISSLQPDDFATYYCQQYDSYVSTFGQGTKVEIKRGGGGSGGGGSGGGGSEVQLLESGGGLVQPGGSLRLSCAASGFTFRNFHVSWVRQAPGKGLEWVSAINGPVGSTYYADSVKGRFTISRDNSKNTLYLQMNSLRAEDTAVYYCAKGYDGFDYWGQGTLVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 63 - protein, 485 aa, synthetic construct
DIQMTQSPSSLSASLGERVSLTCRASQDIGSSLNWLQQEPDGTIKRLIYATSRLDSGVPKRFSGSRSGSDYSLTISSLESEDFVDYYCLQYASSPWTFGGGTKLEIKRGGGGSGGGGSGGGGSQVQLQQPGSVLVRPGASVKLSCKASGYTFTSSWMHWAKQRPGQGLEWIGHIHPNSGISNYNEKFKGKATLTVDTSSSTAYVDLSSLTSEDSAVYYCSRGGRFDDWGAGTTVTVSSGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 64 - RNA, 20 nt, synthetic construct
  source 1..20: mol_type = other RNA
tctctcagctggtacacggc

SEQ ID NO: 65 - RNA, 20 nt, synthetic construct
  source 1..20: mol_type = other RNA
gagaatcaaaatcggtgaat

SEQ ID NO: 66 - RNA, 20 nt, synthetic construct
  source 1..20: mol_type = other RNA
ggccacggagcgagacatct

SEQ ID NO: 67 - RNA, 20 nt, synthetic construct
  source 1..20: mol_type = other RNA
cgcgagcacagctaaggcca